(19) United States
(12) Patent Application Publication
(10) Pub. No.: US 2013/0317291 A1
(43) Pub. Date: Nov. 28, 2013

(54) TREATMENT SYSTEM AND ENDOSCOPE SYSTEM

(71) Applicant: OLYMPUS MEDICAL SYSTEMS CORP., (US)

(72) Inventor: Tetsuya YAMAMOTO, Tokyo (JP)

(73) Assignee: OLYMPUS MEDICAL SYSTEMS CORP., Tokyo (JP)

(21) Appl. No.: 13/757,270

(22) Filed: Feb. 1, 2013

Related U.S. Application Data
(63) Continuation of application No. PCT/JP2012/067592, filed on Jul. 10, 2012.
(60) Provisional application No. 61/506,248, filed on Jul. 11, 2011.

(51) Int. Cl. A61B 17/04; A61B 1/018

(52) U.S. Cl. CPC A61B 17/0469; A61B 1/018
USPC 600/104; 606/147

(57) ABSTRACT

This treatment system includes: a supporting portion; a first grasping member and a second grasping member supported by the supporting portion so as to approach and separate from each other; a needle member including a distal end portion which has a sharp distal end and punctures a tissue, and a proximal end portion continuous with the distal end portion; a holding portion mounted on the first grasping member and releasably holding the proximal end portion of the needle member; and a locking portion mounted on the second grasping member and locking the distal end portion of the needle member such that the proximal end portion of the needle member is removed from the holding portion when the first grasping member and the second grasping member are separated from each other from a position at which the first grasping member and the second grasping member approach each other.

This application is a continuation application based on a PCT Patent Application No. PCT/JP2012/067592, filed on Jul. 10, 2012, whose priority is claimed on U.S. Provisional Patent Application No. 61/506,248, filed on Jul. 11, 2011. The contents of both the PCT Patent Application and the United States Provisional patent application are incorporated by reference herein.

### BACKGROUND OF THE INVENTION

1. Field of the Invention

The present invention relates to a treatment system and an endoscope system for suturing a tissue.

2. Description of Related Art

In the related art, in order to form an opening in a tissue or suture and close the opening formed in the tissue, treatment systems having various configurations are considered.

For example, a suture thread passing forceps disclosed in U.S. Pat. No. 5,730,747 includes a support shaft, and a first holder (a grasping member) and a second holder mounted at an end portion of the support shaft. The first holder is configured to approach and separate from the second holder. Needle holding regions (openings) are formed at distal ends of the first holder and the second holder, respectively.

A needle-attached suture thread used in the suture thread passing forceps has a needle, and a suture thread concentrically attached to the needle. A sharp tip is formed at a portion opposite to a portion of the needle to which the suture thread is attached. The needle-attached suture thread is configured to be detachably attached to a needle holding region of the first holder and a needle holding region of the second holder, respectively.

The needle-attached suture thread is mounted on the needle holding region of the second holder such that the tip of the needle is directed to the first holder. In this state, the tissue is sandwiched between both of the holders, and the first holder approaches the second holder. The needle-attached suture thread moves from the second holder to the first holder. For this reason, the tissue can be sutured by the needle-attached suture thread.

As another example, suture devices disclosed in U.S. Pat. No. 4,164,225 and U.S. Pat. No. 3,470,875 are known. In these suture devices, a lumen (an inner cavity) in which a thread-attached needle can be housed is formed at one of a pair of jaws. The lumen extends in an axial direction of the suture device, is formed in a shape which is bent in a direction in which one of the pair of jaws faces the other of the pair of jaws, and is opened in the one of the pair of jaws toward the other of the pair of jaws. A pusher configured to push the needle out of the opening is disposed in the lumen.

As the tissue is sandwiched between the pair of jaws and the needle is pushed out by the pusher, the tissue can be sutured by the needle.

As still another example, a suture device disclosed in U.S. Pat. No. 4,236,470 is provided. The suture device uses a suture needle having sharp tips formed at both ends thereof. A needle hole and a narrowed portion are formed at a central part of the suture needle. A thread is attached to the needle hole. The suture device includes a pair of jaws that can approach and separate from each other. The jaws are respectively provided with blades which are disposed so as to slide with respect to the jaws. As positions of the blades are adjusted, the blades can engage with the narrowed portion of the suture needle, or this engagement can be released.

In the suture device having the above-mentioned configuration, as the blades are slid to adjust the engagement, the suture needles can be alternately delivered between the pair of jaws, and therefore the tissue can be sutured.

### SUMMARY OF THE INVENTION

According to a first aspect of the present invention, a treatment system includes: a supporting portion provided so as to extend from a distal side to a proximal side thereof; a first grasping member and a second grasping member which are supported by the supporting portion so as to approach and separate from each other, and grasp a tissue; a needle member including a distal end portion which has a sharp distal end and punctures the tissue, and a proximal end portion which is continuous with the distal end portion; a holding portion which is mounted on the first grasping member, releasably holds the proximal end portion of the needle member, and holds the proximal end portion of the needle member such that the distal end portion of the needle member is exposed to an outside when the first grasping member and the second grasping member are separated from each other; and a locking portion which is mounted on the second grasping member such that the locking portion locks the distal end portion of the needle member at a position at which the first grasping member and the second grasping member approach each other, and locks the distal end portion of the needle member such that the proximal end portion of the needle member is removed from the holding portion when the first grasping member and the second grasping member are separated from each other from the position at which the first grasping member and the second grasping member approach each other.

According to a second aspect of the present invention, the treatment system according to the first aspect may further include: a manipulation member which is connected to both the first grasping member and the second grasping member, and freely moves in an extending direction of the supporting portion; a manipulation portion allowing the first grasping member and the second grasping member to approach and separate from each other by the manipulation portion moving the manipulation member; a first puncture member formed so as to project from the first grasping member toward the second grasping member; an abutting portion which is arranged at a distal end portion of the first puncture member; a second puncture member which is arranged at a position opposite to the first puncture member, and is formed so as to project from the second grasping member toward the first grasping member; a blade portion provided at a distal end portion of the second puncture member; and an opening and closing mechanism allowing the blade portion and the abutting portion to be in an abutting state in which the blade portion abuts the abutting portion by the opening and closing mechanism allowing the first grasping member and the second grasping member to approach each other from a position at which the first grasping member and the second grasping member are separated from each other.

According to a third aspect of the present invention, the treatment system according to the second aspect may further include a wire member having flexibility, wherein the proximal end portion of the needle member is mounted on one end of the wire member.

According to a fourth aspect of the present invention, in the treatment system according to the third aspect, a first internal space may be formed in the first puncture member so as to communicate with a first opening formed at a distal end of the first puncture member in a first projecting direction in which the first puncture member projects. A first slit which communicates with the first internal space and into which the wire member is inserted may be formed in at least the distal end portion of the first puncture member in the first projecting direction. A second internal space may be formed in the second puncture member so as to communicate with a second opening formed in a distal end of the second puncture member in a second projecting direction in which the second puncture member projects. A second slit which communicates with the second internal space and into which the wire member is inserted may be formed in both the second grasping member and the second puncture member throughout an entire length in the second projecting direction.

According to fifth aspect of the present invention, the treatment system according to the fourth aspect may further include: an engaging member mounted on one of the first grasping member and the second grasping member; and a shaft-shaped member having elasticity, wherein a proximal end of the shaft-shaped member is connected to an other of the first grasping member and the second grasping member, and a engaged member which is engaged with the engaging member is mounted on a distal end of the shaft-shaped member. When the shaft-shaped member is elastically deformed about the proximal end of the shaft-shaped engaged member and the engaged member engages with the engaging member, the engaged member may engage with the engaging member in both an opposed direction from the proximal end of the shaft-shaped member toward the engaging member and a direction which is substantially perpendicular to the opposed direction and is on a plane surface on which the shaft-shaped member elastically deformed is disposed. The first grasping member and the second grasping member may be separated from each other from the abutting state when the engaged member of the shaft-shaped member elastically deformed engages with the engaging member.

According to a sixth aspect of the present invention, in the fifth aspect, the holding portion may be formed at an inner wall surface which forms the first internal space. The holding portion may generate the holding force by a frictional force generated between the needle member and the holding portion.

According to a seventh aspect of the present invention, in the sixth aspect, the first puncture member and the second puncture member may have tubular shapes. One of the first puncture member and the second puncture member may be inserted into an other of the first puncture member and the second puncture member.

According to an eighth aspect of the present invention, in the seventh aspect, the needle member may include a holding member capable of being held by the holding portion, and a locking member capable of being locked by the locking portion. The holding member may be connected to the locking member and may have an outside diameter smaller than an outer diameter of the locking member. The wire member may include: a thread main body, wherein one end of the thread main body is connected to the holding member, and an outside diameter of the thread main body is set to be smaller than the outside diameter of the locking member; an intermediate portion connected to an other end of the thread main body; and a tissue disposed at an opposite side of the thread main body, wherein one end of the tissue fixing member is connected to the intermediate portion.

According to a ninth aspect of the present invention, in the eighth aspect, the thread main body may be formed of a material having a larger modulus of longitudinal elasticity than the tissue fixing member.

According to a tenth aspect of the present invention, in the ninth aspect, the thread main body may be formed of a metal.

According to an eleventh aspect of the present invention, in the ninth aspect, the tissue fixing member may be formed of a resin having biocompatibility.

According to a twelfth aspect of the present invention, in the eighth aspect, the outside diameter of the thread main body may be smaller than an outside diameter of the tissue fixing member.

According to a thirteenth aspect of the present invention, in the eighth aspect, the locking member may have a substantially hemispherical shape.

According to a fourteenth aspect of the present invention, in the eighth aspect, the intermediate portion and the tissue fixing member may be detachably connected with each other by a detachable connecting structure.

According to a fifteenth aspect of the present invention, in the fourteenth aspect, the detachable connecting structure may include: an insertion hole formed in the intermediate portion; and a knot which is formed at the tissue fixing member, is inserted into the insertion hole, and is tied to the intermediate portion.

According to a sixteenth aspect of the present invention, in the eighth aspect, the intermediate portion and the tissue fixing member may be connected with each other by a fixing connecting structure in which the intermediate portion and the tissue fixing member are fixed with each other and integrated.

According to a seventeenth aspect of the present invention, in the sixteenth aspect, the fixing connecting structure may include: one end of the tissue fixing member; and a connecting concave portion formed at the intermediate portion, wherein the one end of the tissue fixing member is inserted into the connecting concave portion, and side surfaces of the one end of the tissue fixing member is sandwiched by the connecting concave portion.

According to an eighteenth aspect of the present invention, an endoscope system may include: the treatment system according to the first aspect; and an endoscope having a channel into which the treatment system is inserted.

### BRIEF DESCRIPTION OF THE DRAWINGS

FIG. 1 is an overall view of an endoscope system according to a first embodiment of the present invention.

FIG. 2 is a lateral cross-sectional view of a treatment portion in a suture device of the endoscope system according to the first embodiment of the present invention.

FIG. 3 is a perspective view of a second jaw portion of the suture device according to the first embodiment of the present invention.

FIG. 4 is a perspective view of a second puncture member mounted on the second jaw portion of the suture device according to the first embodiment of the present invention.

FIG. 5 is a cross-sectional view taken along section line A1-A1 of FIG. 2.

FIG. 6 is a perspective view of a first puncture member mounted on a first jaw portion of the suture device according to the first embodiment of the present invention.

FIG. 7 is a lateral view of a manipulation portion of the suture device of the first embodiment of the present invention.

FIG. 8 is a lateral cross-sectional view of a wire element of the endoscope system according to the first embodiment of the present invention.

FIG. 9 is a perspective view of an intermediate portion in the wire element according to the first embodiment of the present invention.

FIG. 10 is a lateral cross-sectional view for describing a procedure using the endoscope system according to the first embodiment of the present invention.

FIG. 11 is a cross-sectional view of a major part for describing the procedure using the endoscope system according to the first embodiment of the present invention.

FIG. 12 is a lateral cross-sectional view for describing the procedure using the endoscope system according to the first embodiment of the present invention.

FIG. 13 is a lateral cross-sectional view for describing the procedure using the endoscope system according to the first embodiment of the present invention.

FIG. 14 is a cross-sectional view taken along section line A2-A2 of FIG. 13.

FIG. 15 is a lateral cross-sectional view for describing the procedure using the endoscope system according to the first embodiment of the present invention.

FIG. 16 is a lateral cross-sectional view for describing the procedure using the endoscope system according to the first embodiment of the present invention.

FIG. 17 is a cross-sectional view of a major part for describing the procedure using the endoscope system according to the first embodiment of the present invention.

FIG. 18 is a view for describing the procedure using the endoscope system according to the first embodiment of the present invention.

FIG. 19 is a cross-sectional view of a fixing mechanism of a modified example of the endoscope system according to the first embodiment of the present invention.

FIG. 20 is a lateral cross-sectional view of a modified example of the endoscope system according to the first embodiment of the present invention.

FIG. 21 is a cross-sectional view of a major part of a modified example of the endoscope system according to the first embodiment of the present invention.

FIG. 22 is a cross-sectional view of a major part of a modified example of the endoscope system of the first embodiment of the present invention.

FIG. 23 is a cross-sectional view of a major part of a modified example of the endoscope system according to the first embodiment of the present invention.

FIG. 24 is a cross-sectional view of a major part of a modified example of the endoscope system according to the first embodiment of the present invention.

FIG. 25 is a cross-sectional view for describing a motion of a modified example of the endoscope system according to the first embodiment of the present invention.

FIG. 26 is a cross-sectional view for describing the motion of the modified example of the endoscope system according to the first embodiment of the present invention.

FIG. 27 is a cross-sectional view of a wire element of a modified example of the endoscope system according to the first embodiment of the present invention.

FIG. 28 is an overall view of a wire element of a modified example of the endoscope system according to the first embodiment of the present invention.

FIG. 29 is a cross-sectional view of the wire element of the modified example of the endoscope system of the first embodiment of the present invention.

FIG. 30 is a lateral cross-sectional view of a treatment portion in a suture device of an endoscope system according to a second embodiment of the present invention.

FIG. 31 is a partially cutaway plan view showing the suture device of the endoscope system according to the second embodiment of the present invention.

FIG. 32 is a cross-sectional view taken along section line A3-A3 of FIG. 30.

FIG. 33 is a lateral view of a manipulation portion of the suture device of the endoscope system according to the second embodiment of the present invention.

FIG. 34 is a lateral view of a major part for describing a procedure using the endoscope system according to the second embodiment of the present invention.

FIG. 35 is a partially cutaway plan view for describing the procedure using the endoscope system according to the second embodiment of the present invention.

FIG. 36 is a lateral cross-sectional view of an endoscope system according to a third embodiment of the present invention.

FIG. 37 is a cross-sectional view taken along section line A4-A4 of FIG. 36.

FIG. 38 is a perspective view showing a second puncture member of a modified example of the endoscope system of the first embodiment of the present invention.

### DETAILED DESCRIPTION OF THE INVENTION

### First Embodiment

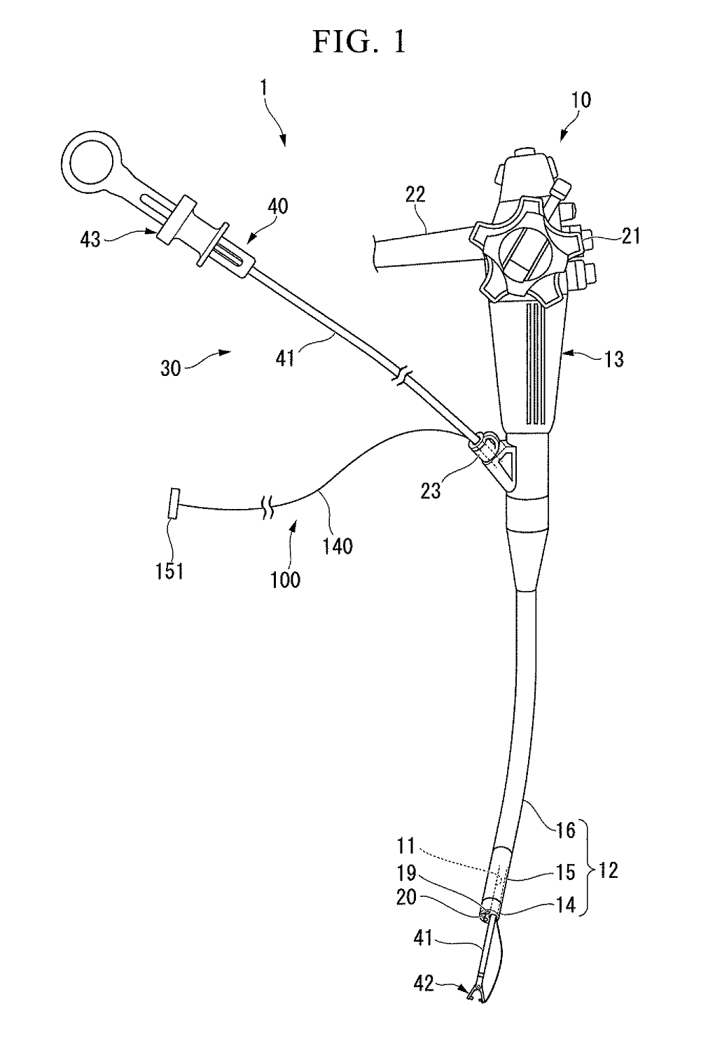

Hereinafter, an endoscope system according to a first embodiment of the present invention is described referring to FIGS. 1 to 29. The endoscope system performs treatment of suturing a tissue in a suture system (a treatment system) while observing the inside of a body cavity with an endoscope.

As shown in FIG. 1, an endoscope system 1 includes an endoscope 10, and a suture system 30. The endoscope 10 has a forceps channel (a channel) 11 formed therein. The suture system 30 has an insertion portion 41 that can be inserted into the forceps channel 11.

The endoscope 10 uses a well-known configuration. The endoscope 10 includes a long endoscope insertion portion 12, and an endoscope manipulation portion 13. The endoscope manipulation portion 13 is connected to a proximal end portion of the endoscope insertion portion 12. The endoscope insertion portion 12 includes a distal end rigid portion 14, a bending portion 15 which can be bent, and a flexible tube portion 16. The distal end rigid portion 14 is provided at a distal end of the endoscope insertion portion 12. The bending portion 15 is connected to a proximal end of the distal end rigid portion 14. The flexible tube portion 16 is connected to a proximal end of the bending portion 15.

A lighting unit 19 and an observation unit 20 are provided at a front surface of the distal end rigid portion 14. The lighting unit 19 is a fiberglass, an LED, or the like, configured to emit illumination light. The observation unit 20 has a plurality of optical lenses and a CCD which are configured to detect the reflected illumination light. In addition, the observation unit 20 converts the detected illumination light into an image signal and transmits the image signal to the endoscope manipulation portion 13.

A manipulation dial 21 configured to manipulate the bending portion 15 such that the bending portion 15 is bent is mounted on the endoscope manipulation portion 13. One end of a universal cable 22 is connected to the endoscope manipulation portion 13. The other end of the universal cable 22 is connected to a display unit (not shown). The display unit can convert the image signal converted by the observation unit 20 into an image and display the image.

The above-mentioned forceps channel 11 communicates with an opening formed in the front surface of the distal end rigid portion 14. The forceps channel 11 extends to the endoscope manipulation portion 13 through the inside of the endoscope insertion portion 12, and communicates with an opening of a forceps stopper 23 mounted on the endoscope manipulation portion 13.

The suture system 30 according to the present embodiment includes a suture device 40 and a wire element 100. The suture device 40 sutures the tissue. The wire element 100 is configured to be detachably attached to the suture device 40.

The suture device 40 includes the above-mentioned long insertion portion 41, a treatment portion 42, and a manipulation portion 43. The treatment portion 42 is mounted on a distal end of the insertion portion 41. The manipulation portion 43 is mounted on a proximal end of the insertion portion 41 and manipulates the treatment portion 42. Hereinafter, first, the treatment portion 42 is described.

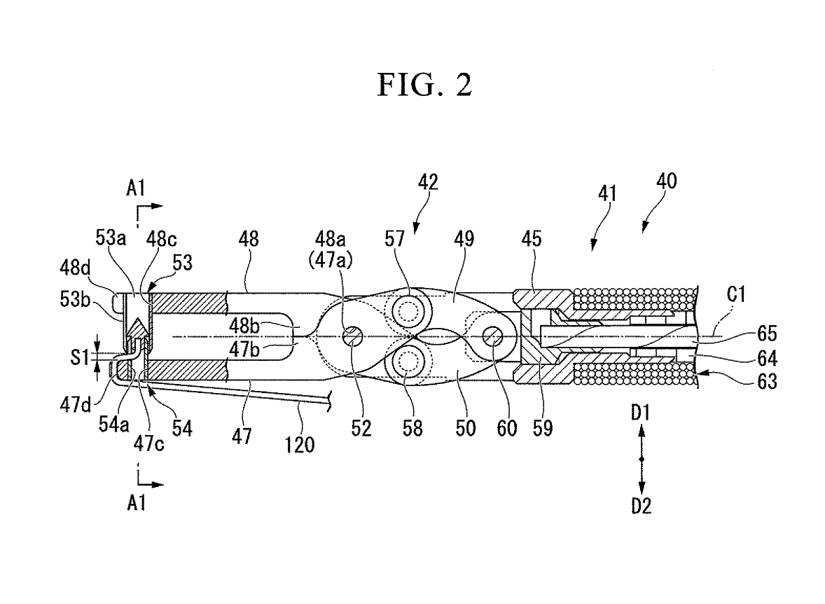

As shown in FIG. 2, the treatment portion 42 has a substantially cylindrical base member (a supporting portion) 45, a first jaw portion (a first grasping member) 47, a second jaw portion (a second grasping member) 48, and coupling members 49 and 50. The first jaw portion 47, the second jaw portion 48, and the coupling members 49 and 50 are connected to the base member 45. The treatment portion 42 is formed of a metal such as stainless steel in order to secure a certain level of water resistance and strength.

The base member 45 lengthily extends in a direction from a distal side to a proximal side thereof. An axis C1 (an extending direction) is set to this direction.

The jaw portions 47 and 48 are disposed in a position in front of the base member 45 so as to extend in the axis C1 direction of the base member 45. A through hole 47a is formed in a direction perpendicular to the axis C1 direction at an intermediate portion of the first jaw portion 47 in the axis C1 direction. A through hole 48a is formed in the direction perpendicular to the axis C1 direction at an intermediate portion of the second jaw portion 48 in the axis C1 direction. A hinge pin 52 fixed to the base member 45 is inserted into the through hole 47a of the first jaw portion 47 and the through hole 48a of the second jaw portion 48. As a result, the first jaw portion 47 and the second jaw portion 48 are supported by the hinge pin 52 so as to rotate about the hinge pin 52.

The first jaw portion 47 is provided with a first abutting member 47b which is formed closer to a distal side of the first jaw portion 47 than the through hole 47a. The first abutting member 47b is formed at a first projecting direction D1 side, in which a first puncture member 54 (to be described below) projects, in the first jaw portion 47. Similarly, the second jaw portion 48 is provided with a second abutting member 48b which is formed closer to a distal side of the second jaw portion 48 than the through hole 48a. The second abutting member 48b is formed at a second projecting direction D2 side, in which a second puncture member 53 (to be described below) projects, in the second jaw portion 48. That is, the second abutting member 48b is formed at an opposite side of the above-mentioned first projecting direction D1.

When jaw portions 47 and 48 rotate about the hinge pin 52 and a distal end of the first jaw portion 47 and a distal end of the second jaw portion 48 approach each other to a certain distance, the jaw portions 47 and 48 reach an abutting state (to be described below). Here, the first abutting member 47b and the second abutting member 48b abut each other. For this reason, the distal ends of the jaw portions 47 and 48 are prevented from further approaching each other.

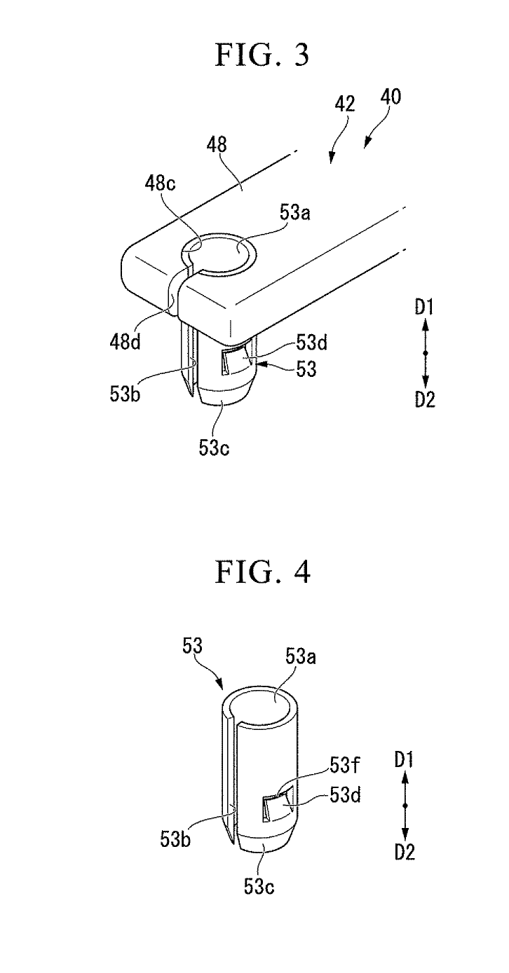

As shown in FIGS. 2 and 3, a fixing hole 48c penetrating the second jaw portion 48 in the second projecting direction D2 is formed in a distal end portion of the second jaw portion 48. A second jaw portion side slit 48d which communicates with the fixing hole 48c and extends to the distal end portion is formed in the second jaw portion 48. The second jaw portion side slit 48d penetrates the second jaw portion 48 in the second projecting direction D2. The second jaw portion side slit 48d has a slit width set such that a thread main body 120 (to be described below) of the wire element 100 can be inserted thereinto.

The second puncture member 53 formed in a tubular shape is fixed to the fixing hole 48c. In order to reduce the load applied to a living body, an inside diameter of the second puncture member 53 is set to, for example, about 0.5 mm. In the second puncture member 53, a duct line 53a corresponds to a second inner cavity (a second internal space). In the second puncture member 53, a second opening is formed in an end portion of the duct line 53a in the second projecting direction D2. In the suture device 40 according to the present embodiment, a second inner cavity is formed so as to penetrate the second puncture member 53 in the second projecting direction D2.

As described above, a portion of the second protruding direction D2 side of the second puncture member 53 projects toward the second projecting direction D2 side from the second jaw portion 48. As shown in FIG. 2, a length at which the second puncture member 53 projects in the second projecting direction D2 is a length such that a gap S1 is left between a distal end of the projecting portion of the second puncture member 53 and the first jaw portion 47. When the jaw portions 47 and 48 are in the abutting state, the gap S1 is adjusted such that the thread main body 120 (to be described below) of the wire element 100 can be inserted between a distal end of the second puncture member 53 in the second projecting direction D2 and the first jaw portion 47 (see FIG. 2).

On the other hand, a portion of the first projecting direction D1 side of the second puncture member 53 is set to be disposed on the same plane as a surface of the first projecting direction D1 side of the second jaw portion 48. The second puncture member 53 is formed so as to penetrate the second jaw portion 48.

As shown in FIGS. 3 and 4, a second puncture member side slit 53b communicating with the duct line 53a is formed in a side surface of the second puncture member 53 throughout the entire length of the second puncture member 53. A width of the second puncture member side slit 53b is set to be substantially equal to the width of the second jaw portion side slit 48d. The second puncture member 53 is disposed in the fixing hole 48c of the second jaw portion 48 such that the second puncture member side slit 53b communicates with the second jaw portion side slit 48d.

The second jaw portion side slit 48d and the second puncture member side slit 53b correspond to a second slit.

A blade portion 53c is formed at the distal end of the second puncture member 53 in the second projecting direction D2, i.e., an edge portion of the second opening of the duct line 53a, throughout the entire circumference except for a portion at which the second puncture member side slit 53b is formed. A distal end of the blade portion 53c has the same shape in a circumferential direction thereof.

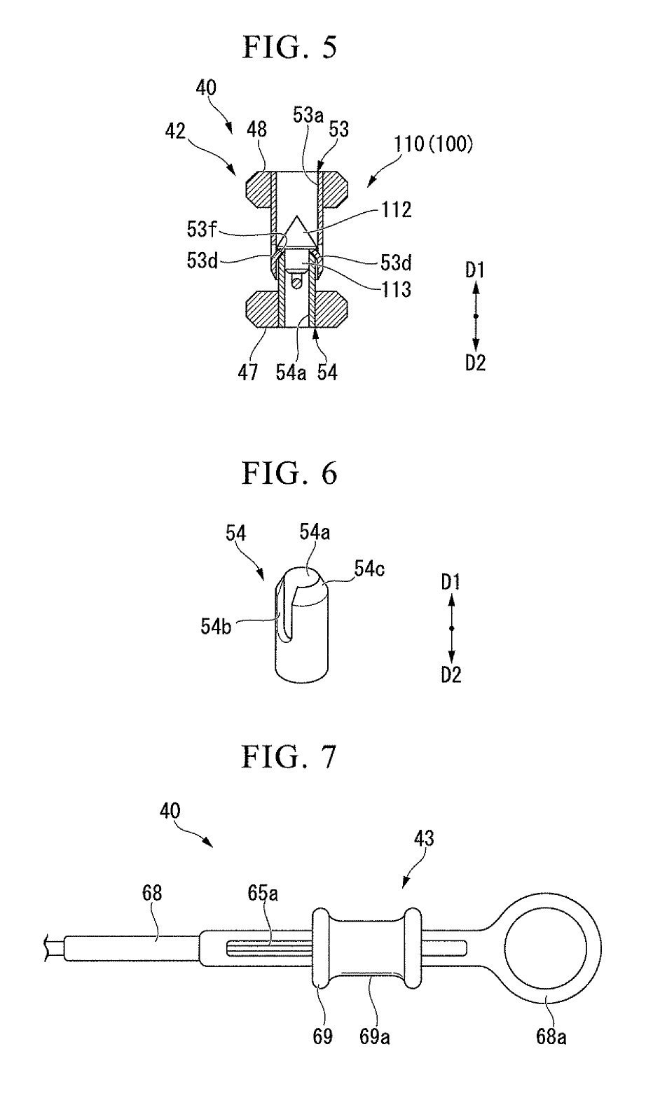

As shown in FIGS. 4 and 5, a pair of tongue portions (locking portions) 53d extending toward the first projecting direction D1 side in a duct line 53a side is formed at the second puncture member 53. The pair of tongue portions 53d is disposed at a position symmetrical about an axis of the second puncture member 53 (see FIG. 5).

For example, the tongue portions 53d can be formed as follows. First, the side surface of the second puncture member 53 is cut out in substantially a U shape. Next, the cutout portion is plastically deformed toward the duct line 53a.

As shown in FIG. 2, a fixing hole 47c penetrating the first jaw portion 47 in the first projecting direction D1 is formed in a distal end portion of the first jaw portion 47 at a position opposite to the fixing hole 48c of the second jaw portion 48. A bypass slit 47d which communicates with the fixing hole 47c and penetrates the first jaw portion 47 in the first projecting direction D1 is formed in a distal end surface of the first jaw portion 47. The width of the bypass slit 47d is set such that the thread main body 120 of the wire element 100 can be inserted into the bypass slit 47d.

As shown in FIGS. 2 and 6, the first puncture member 54 formed in a tubular shape is fixed to the fixing hole 47c. An opening at the first projecting direction D1 side in a duct line (a first internal space) 54a of the first puncture member 54 is disposed so as to be opposite to the second opening at the second projecting direction D2 side in the duct line 53a of the second puncture member 53. An outside diameter of the first puncture member 54 is set to be slightly smaller than an inside diameter of the second puncture member 53 such that the first puncture member 54 can be inserted into the duct line 53a of the second puncture member 53.

As described above, a portion of the first projecting direction D1 side of the first puncture member 54 projects toward the first projecting direction D1 side from the first jaw portion 47. A length at which the first puncture member 54 projects in the first projecting direction D1 is adjusted so as to satisfy the following two conditions when the jaw portions 47 and 48 are in the abutting state. As shown in FIG. 5, a first condition is that the duct line 53a and the duct line 54a are in a communication state in which the duct line 53a communicates with the duct line 54a. A second condition is that, as a distal end portion of the first puncture member 54 in the first projecting direction D1 is press-fitted in between the pair of tongue portions 53d, the needle member 110 (to be described below) of the wire element 100 is locked by the second puncture member 53. For this reason, the needle member 110 can be prevented from moving from the inside of the second puncture member 53 to the inside of the first puncture member 54.

On the other hand, as shown in FIG. 2, a portion of the second projecting direction D2 side of the first puncture member 54 is set to be disposed on the same plane as a surface of the second projecting direction D2 side of the first puncture member 54.

As shown in FIG. 6, a first puncture member side slit (a first slit) 54b communicating with the duct line 54a is formed in a side surface of the first puncture member 54 throughout a predetermined range from an end portion of the first puncture member 54 in the first projecting direction D1. The width of the first puncture member side slit 54b is set to be substantially equal to the width of the second puncture member side slit 53b. The first puncture member side slit 54b is disposed at a distal side of the first puncture member 54. A blade portion (an abutting portion) 54c is formed at the distal end of the first puncture member 54 in the first projecting direction D1 throughout the entire circumference except for a portion at which the first puncture member side slit 54b is formed.

The first puncture member side slit 54b is formed in the first puncture member 54. For this reason, the distal end portion of the first puncture member 54 in the first projecting direction D1 can be easily deformed so as to be reduced in diameter. As a result, press-fitting of the first puncture member 54 becomes easy.

When the jaw portions 47 and 48 are in the abutting state, the blade portion 53c of the second puncture member 53 abuts (slidingly contacts) the blade portion 54c. Here, as described below, a tissue sandwiched between the blade portion 54c and the blade portion 53c is cut.

As shown in FIG. 2, a proximal side of the first jaw portion 47 and a distal side of a coupling member 49 are rotatably connected by a coupling pin 57. Similarly, a proximal side of the second jaw portion 48 and a distal side of a coupling member 50 are rotatably connected by a coupling pin 58.

A proximal side of the coupling member 49 and a proximal side of the coupling member 50 are supported by a hinge pin 60 fixed to a connecting member 59 so as to rotate about the hinge pin 60. The connecting member 59 is configured to slidee with respect to the base member 45 in the axis C1 direction.

The jaw portions 47 and 48, and the coupling members 49 and 50 having the above-mentioned configuration constitute a so-called pantograph type link mechanism. In the above-mentioned configuration, as the connecting member 59 is slid in the axis C1 direction, the jaw portions 47 and 48 can approach and separate from each other.

The insertion portion 41 includes a long outer sheath 63, an inner sheath 64, and a manipulation wire (a manipulation member) 65. The outer sheath 63 extends in the axis C1 direction. The inner sheath 64 is inserted into the outer sheath 63. The manipulation wire 65 is inserted into the inner sheath 64. A distal end of the manipulation wire 65 is fixed to the connecting member 59.

In the suture device 40 according to the present embodiment, a three-layer coil (a multi-layer coil) is used as the outer sheath 63. The multi-layer coil can efficiently transmit torque applied to a proximal side of the insertion portion 41 around the axis C1 to a distal side of the insertion portion 41.

A single layer flat coil is used as the inner sheath 64. The flat coil can effectively transmit a compression force applied to the proximal side of the insertion portion 41 in the axis C1 direction to the distal side of the insertion portion 41.

The distal sides of the outer sheath 63 and the inner sheath 64 are fixed to the base member 45.

A distal end of the manipulation wire 65 is fixed to the coupling members 49 and 50 via the connecting member 59. The manipulation wire 65 can move in a duct line of the inner sheath 64 in the axis C1 direction.

The base member 45, the coupling members 49 and 50, and the connecting member 59 correspond to an opening and closing mechanism.

As shown in FIG. 7, the manipulation portion 43 has a rod shape. The manipulation portion 43 has a manipulation portion main body 68 and a wire slider 69. A distal end of the manipulation portion main body 68 is connected to proximal ends of the outer sheath 63 and the inner sheath 64. The wire slider 69 is mounted on an outer circumferential surface of the manipulation portion main body 68 so as to be able to slide in a longitudinal direction of the manipulation portion main body 68. For example, a finger hooking ring 68a in which an operator's thumb can be put is formed at a proximal end of the manipulation portion main body 68.

A depression 69a is formed on an outer circumferential surface of the wire slider 69. A proximal end of a stainless steel pipe 65a through which the manipulation wire 65 is inserted is connected to the wire slider 69.

In the suture device 40 configured as described above, the depression 69a is pinched between the operator's index finger and middle finger and the operator's thumb is put into the finger hooking ring 68a. Then, as the wire slider 69 is slid with respect to the manipulation portion main body 68, the manipulation wire 65 moves along the axis C1. As a result, the opening and closing mechanism is manipulated, so that the jaw portions 47 and 48 rotates about the hinge pin 52, and the distal ends of the jaw portions 47 and 48 can approach and separate from each other.

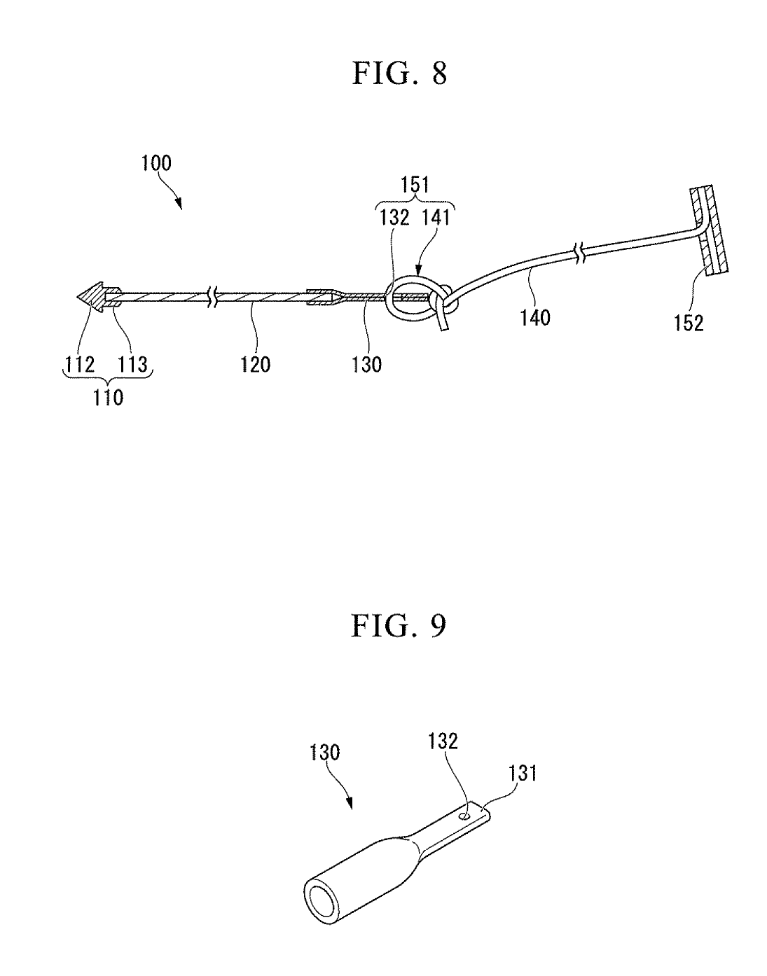

As shown in FIG. 8, the wire element 100 has a needle member 110, the thread main body 120, an intermediate portion 130, and a tissue fixing member 140. A first end of the thread main body 120 is connected to the needle member 110. A second end of the thread main body 120 is connected to the intermediate portion 130. A first end of the tissue fixing member 140 is connected to the intermediate portion 130. Furthermore, the tissue fixing member 140 is disposed on an opposite side of the intermediate portion 130 from the thread main body 120. The thread main body 120, the intermediate portion 130, and the tissue fixing member 140 correspond to the wire member.

The needle member 110 is formed of a metal such as stainless steel, titanium, a titanium alloy, or the like. The needle member 110 has a locking member 112 and a holding member 113. The locking member 112 has a substantially conical shape. The holding member 113 is formed at a bottom surface of the locking member 112.

The holding member 113 has a substantially cylindrical shape. The first end of the thread main body 120 is fixed to a duct line of the holding member 113 through brazing, soldering, laser welding, caulking, or the like.

In the present embodiment, the locking member 112 and the holding member 113 are integrally formed with each other.

As shown in FIG. 5, the needle member 110 is formed so as to be inserted into the duct line 53a of the second puncture member 53. An outside diameter of the holding member 113 is set such that the holding member 113 can be inserted between the pair of tongue portions 53d in a natural state. An outside diameter of the locking member 112 (an outside diameter of the bottom surface side of the locking member 112) is set such that the locking member 112 can be inserted between the tongue portions 53d when the pair of tongue portions 53d are deformed so as to be separated from each other. The outside diameter of the locking member 112 is set such that the locking member 112 cannot be inserted between the tongue portions 53d in a normal undeformed state.

The outside diameter of the holding member 113 is set such that an appropriate frictional force (holding force) is generated in the second projecting direction D2 between an outer circumferential surface of the holding member 113 and an inner wall surface of the duct line 54a when the holding member 113 inserted into the duct line 54a of the first puncture member 54 is removed in the first projecting direction D1. For this reason, the needle member 110 is configured such that the needle member 110 is prevented from being easily removed from the duct line 54a. In order to accomplish the above-mentioned configuration, for example, an inside diameter of the first puncture member 54 may be adjusted to be substantially equal to or slightly larger than the outside diameter of the holding member 113. In addition, the above-mentioned frictional force may be generated by forming a minute concavo-convex shape in the inner wall surface of the duct line 54a.

When the holding member 113 is inserted into the duct line 54a of the first puncture member 54, a bottom surface of the locking member 112 is locked by the end portion of the first puncture member 54 in the first projecting direction D1.

As described above, the needle member 110 is formed to be detachably attached to the first puncture member 54 and the second puncture member 53, respectively.

As shown in FIG. 8, an outside diameter of the thread main body 120 is set to be smaller than an outside diameter of the needle member 110, i.e., the outside diameter of the locking member 112. Furthermore, the outside diameter of the thread main body 120 is set to be smaller than an outside diameter of the tissue fixing member 140. The thread main body 120 is formed of a material having a larger modulus of longitudinal elasticity than the tissue fixing member 140. Specifically, the thread main body 120 is formed of a metal such as stainless steel, titanium, a titanium alloy, or the like.

As shown in FIG. 9, the intermediate portion 130 has a substantially cylindrical shape. An end portion 131 of the intermediate portion 130, which is connected to the first end of the tissue fixing member, is formed in a shape in which a tube is squashed. An insertion hole 132 penetrating the end portion 131 in a thickness direction of the intermediate portion 130 is formed in an end portion 131 side of the intermediate portion 130.

The tissue fixing member 140 is formed of a resin having biocompatibility such as a polyimide, a polyether ether ketone, a polysulfone, a polyamide, or the like. As shown in FIG. 8, the intermediate portion 130 and the first end of the tissue fixing member 140 are releasably connected by a detachable connecting structure 151. In the present embodiment, the detachable connecting structure 151 includes the insertion hole 132 of the intermediate portion 130, and a knot 141 formed at the first end of the tissue fixing member 140, inserted into the insertion hole 132, and tied to the intermediate portion 130. As the first end of the tissue fixing member 140 forms the knot 141 through the insertion hole 132, the tissue fixing member 140 can be easily mounted on the intermediate portion 130. In addition, as the formed knot 141 is untied, the tissue fixing member 140 can be easily separated from the intermediate portion 130.

In the wire element 100 according to the present embodiment, a bar 152 disposed perpendicular to the tissue fixing member 140 is mounted on a second end of the tissue fixing member 140. Depending on a procedure performed using the endoscope system 1 according to the present embodiment, the bar 152 may not be provided in the wire element 100.

Next, the procedure using the endoscope system 1 according to the present embodiment is described. Hereinafter, the case in which an opening formed in a stomach wall (a tissue) is sutured is described. However, a target area is not limited thereto. For example, the target area may be a hollow organ such as the esophagus, the duodenum, the small intestine, the large intestine, the uterus, the bladder, or the like. In addition, a natural opening into which the endoscope 10 is inserted is not limited to the mouth but may be the nose or the anus. Furthermore, the procedure may be used for curing or hemostasis of a punctured area due to an ulcer, plication of a defective area of a mucous membrane, a diverticulum of the digestive tract, or the like.

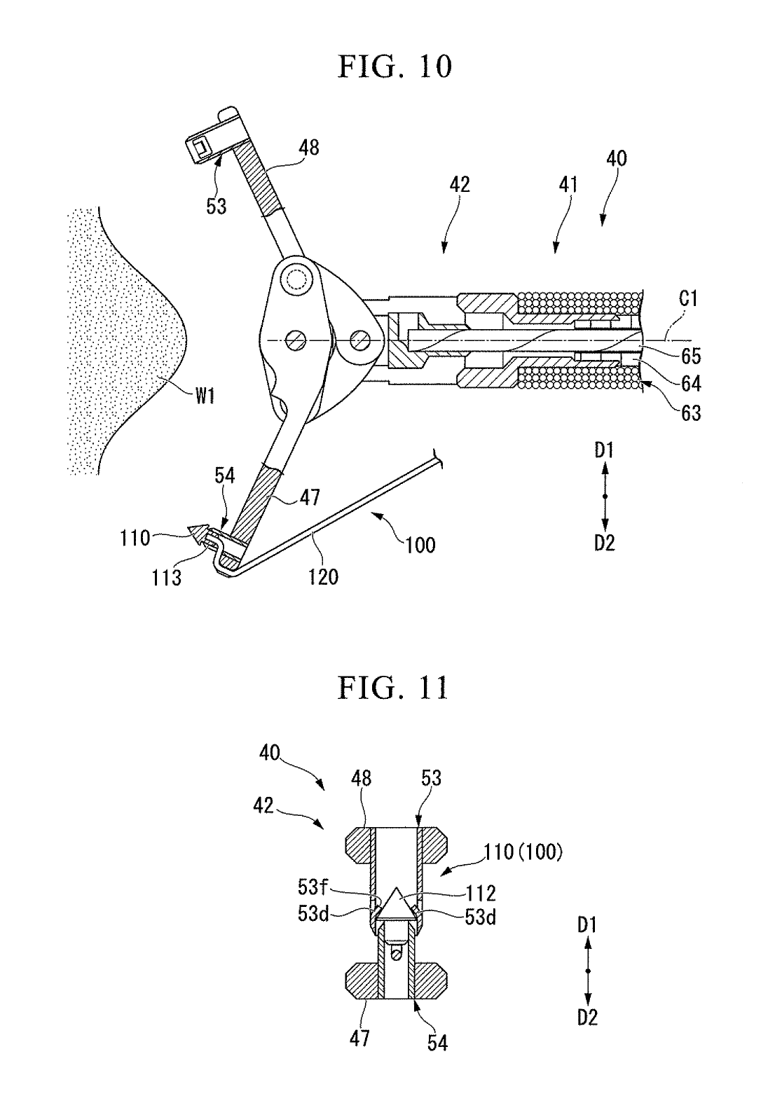

The operator slides the wire slider 69 to the distal side with respect to the manipulation portion main body 68 at the outside of the body of the patient, and as shown in FIG. 10, separates the jaw portions 47 and 48 from each other (an open state). The holding member 113 of the needle member 110 is inserted into the first puncture member 54. The holding member 113 is held at the first puncture member 54 by the frictional force between the first puncture member 54 and the holding member 113. For this reason, the wire element 100 is mounted on the first puncture member 54. Here, the thread main body 120 is inserted into the first puncture member side slit 54b of the first puncture member 54 and the bypass slit 47d of the first jaw portion 47. The thread main body 120 is drawn from a second projecting direction D2 side of the first jaw portion 47 to a proximal side of the suture device 40. The wire slider 69 is slid to the proximal side, and as shown in FIG. 11, the locking member 112 of the wire element 100 is press-fitted into the pair of tongue portions 53d. The first jaw portion 47 and the second jaw portion 48 are locked. Here, the locking member 112 is not locked to the end faces 53f of the tongue portions 53d.

Accordingly, in a state in which the wire element 100 is mounted on the first puncture member 54, the widths of the first jaw portion 47 and the second jaw portion 48 in the first projecting direction D1 are reduced. For this reason, the treatment portion 42 is easily inserted into the forceps channel 11 of the endoscope 10.

Next, the insertion portion 41 of the suture device 40 is inserted into the forceps channel 11 of the endoscope 10, and the suture device 40 is mounted on the endoscope 10. Here, the distal end of the insertion portion 41 does not project forward from the forceps channel 11. The suture device 40 is inserted into the stomach from the mouth of the patient together with the endoscope 10. An external channel may be mounted on the endoscope 10, and the suture device 40 may be inserted into the stomach via the external channel.

The manipulation dial 21 is appropriately manipulated to bend the bending portion 15 while the front of the endoscope insertion portion 12 is observed with the display unit (not shown), and the endoscope insertion portion 12 is inserted.

As the distal end of the endoscope insertion portion 12 arrives at a position opposite to the opening, the insertion portion 41 of the suture device 40 is pushed into the forceps channel 11. As shown in FIG. 10, the treatment portion 42 projects forward from the forceps channel 11. The wire slider 69 is slid to the distal side, and the jaw portions 47 and 48 are opened.

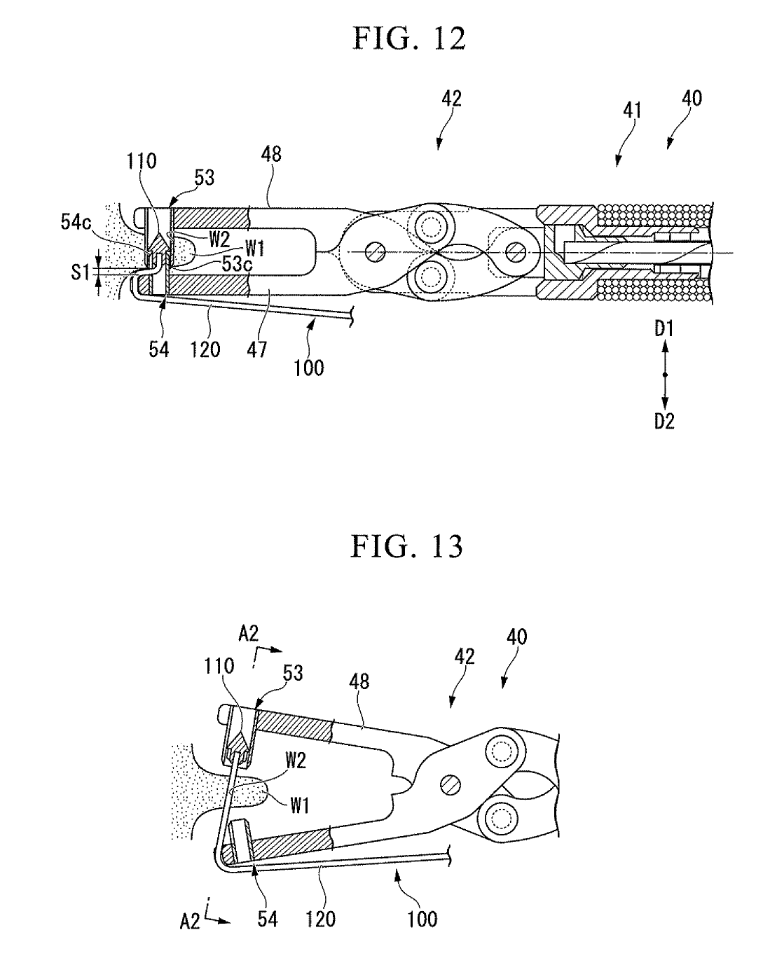

One edge portion W1 of the opening is disposed between the first jaw portion 47 and the second jaw portion 48. In this state, as the wire slider 69 is slid to the proximal side, as shown in FIG. 12, the jaw portions 47 and 48 abut each other (the abutting state).

When the locking member 112 of the wire element 100 enters the inside of the second puncture member 53, as shown in FIG. 11, the locking member 112 is already press-fitted into the pair of tongue portions 53d. Here, since the pair of tongue portions 53d is deformed outward along the outer circumferential surface of the locking member 112, as shown in FIG. 5, the locking member 112 can be inserted between the tongue portions 53d.

Here, since the locking member 112 is locked by the tongue portion 53d, the needle member 110 cannot exit to the second projecting direction D2 side with respect to the second puncture member 53. As a result, the wire element 100 is mounted on the second puncture member 53 against the frictional force between the first puncture member 54 and the holding member 113.

As described above, when the jaw portions 47 and 48 are in the abutting state, the wire element 100 mounted on the first puncture member 54 is mounted on the second puncture member 53 to deliver the wire element 100.

As shown in FIG. 12, as the second puncture member 53 communicates with the first puncture member 54, the blade portion 53c of the second puncture member 53 abuts the blade portion 54c of the first puncture member 54. As a result, a through hole W2 is formed in the one edge portion W1 by a shearing force between the blade portion 54c and the blade portion 53c. Since the blade portions 53c and 54c are formed, the puncture members 53 and 54 can penetrate the one edge portion W1.

As described above, the gap S1 is formed between the distal end of the second puncture member 53 in the second projecting direction D2 and the first jaw portion 47. For this reason, the thread main body 120 of the wire element 100 is prevented from being cut by the blade portion 53c of the second puncture member 53.

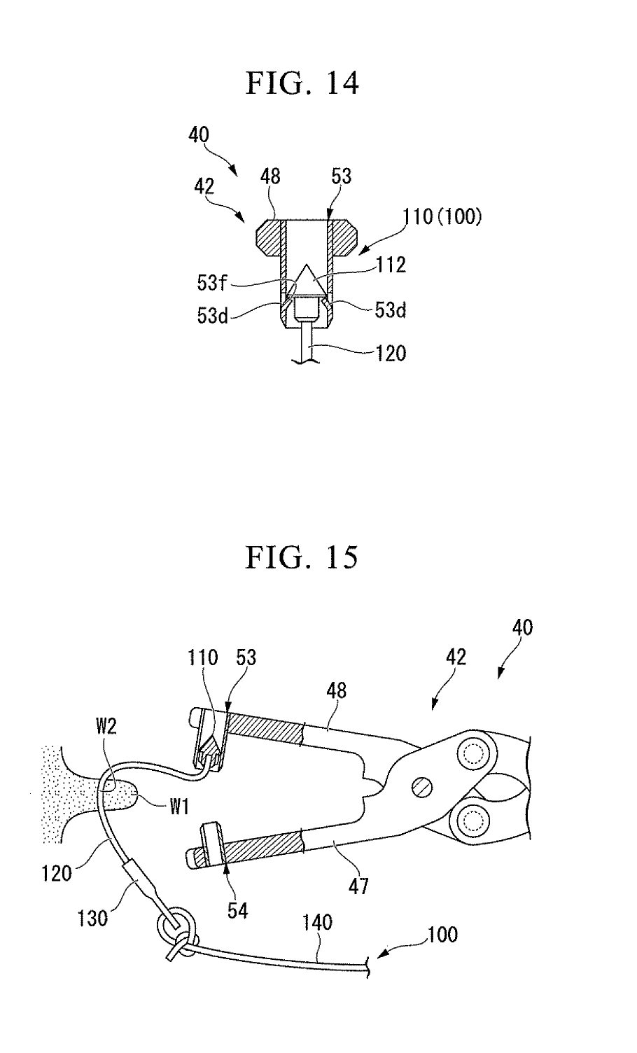

Here, as the jaw portions 47 and 48 are opened, as shown in FIG. 13, the jaw portions 47 and 48 are separated from each other in a state in which the needle member 110 is mounted on the second puncture member 53. Here, the thread main body 120 is inserted into the through hole W2. As shown in FIG. 14, the locking member 112 of the needle member 110 is locked by the tongue portions 53d.

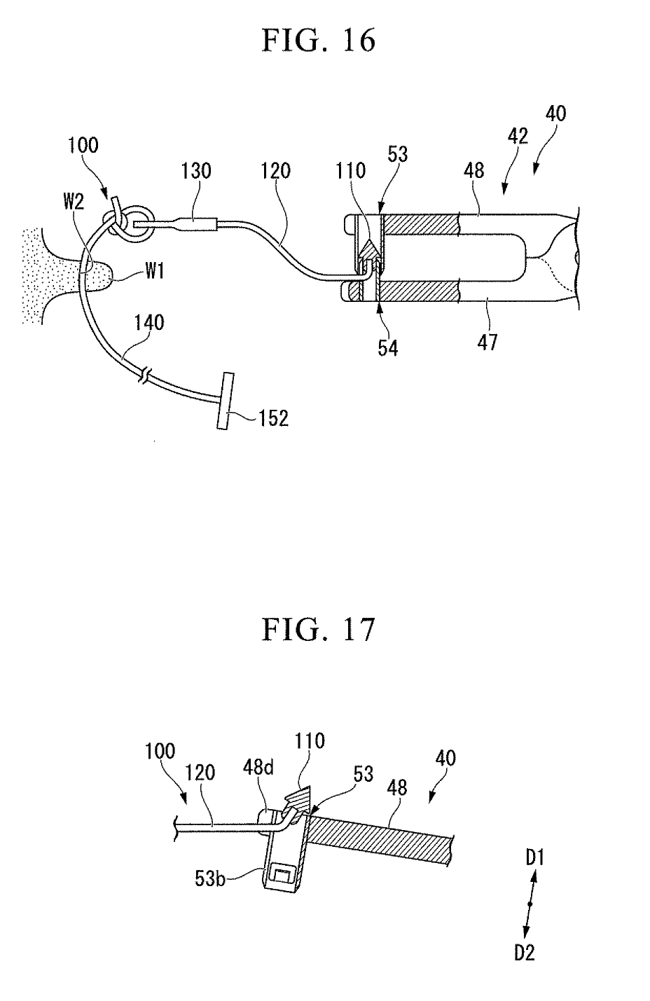

As a portion of the insertion portion 41 of the suture device 40 is retracted to the forceps channel 11 or the entire endoscope 10 is moved, as show in FIG. 15, the thread main body 120 moves in the through hole W2. Here, since the needle member 110 is prevented from being removed from the second puncture member 53 due to carelessness, the jaw portions 47 and 48 may be lightly closed. In addition, as the above-mentioned operation is further performed, as shown in FIG. 16, the intermediate portion 130 passes through the through hole W2, and the tissue fixing member 140 is inserted into the through hole W2. After the tissue fixing member 140 is inserted into the one edge portion W1, when the suture device 40 is removed from the forceps channel 11, one stitch at the one the edge portion W1 by the tissue fixing member 140 is done.

As shown in FIG. 17, as the thread main body 120 is inserted into the second puncture member side slit 53b and the second jaw portion side slit 48d while the needle member 110 is moved toward the first projecting direction D1 side with respect to the second puncture member 53, the wire element 100 is removed from the second puncture member 53.

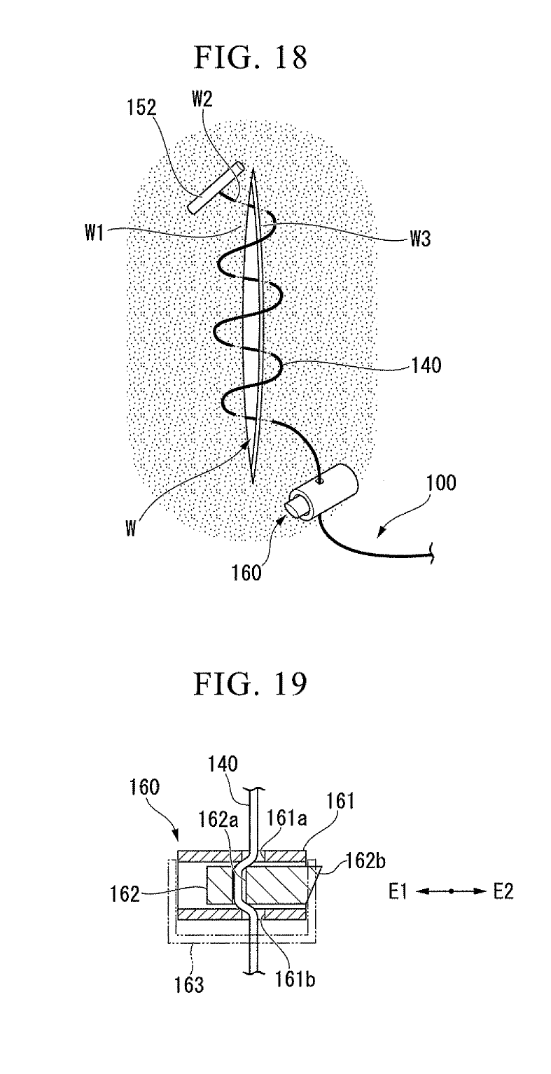

After that, as described above, the operator mounts the needle member 110 on the first puncture member 54. The insertion portion 41 is inserted into the forceps channel 11 again, and as shown in FIG. 18, the other edge portion W3 of the opening W is similarly sutured by one stitch. As described above, as the one edge portion W1 and the other edge portion W3 are alternately sutured, the opening W can be sutured.

As a fixing mechanism 160 is mounted on an intermediate portion of the tissue fixing member 140, the tissue fixing member 140 can be prevented from being removed from the sutured edge portions W1 and W3. As shown in FIG. 19, the fixing mechanism 160 is constituted by a main body 161 having a cylindrical shape, and a lock member 162 having a substantially columnar shape and configured to be inserted into the main body 161.

A pair of through holes 161a and 161b is formed in a side surface of the main body 161 on the same straight line to sandwich a duct line of the main body 161. A through hole 162a is formed in the lock member 162 in a radial direction. As the lock member 162 is moved in an axial direction of the main body 161 with respect to the main body 161, the through holes 161a and 161b and the through hole 162a are disposed on the same straight line. Here, the tissue fixing member 140 can be inserted into the through holes 161a and 161b and the through hole 162a.

In a state in which the tissue fixing member 140 is inserted into the through holes 161a, 161b and 162a, the lock member 162 is moved with respect to the main body 161 in a direction E1 parallel to the axis of the main body 161. For this reason, the frictional force can be applied between the tissue fixing member 140 and both the main body 161 and the lock member 162, and the fixing mechanism 160 can be fixed to the tissue fixing member 140.

In order to move the lock member 162 with respect to the main body 161 in the direction E1, a sealing member 163 may be provided. A position of the fixing mechanism 160 is held with respect to the tissue fixing member 140 in a direction in which the tissue fixing member 140 passes. In this state, as the sealing member 163 is slidingly contacted with a slope portion 162b of the lock member 162, the lock member 162 moves in the direction E1. As a result, the fixing mechanism 160 is fixed to the tissue fixing member 140.

In the suture devices disclosed in U.S. Pat. No. 5,730,747, U.S. Pat. No. 4,164,225, U.S. Pat. No. 3,470,875, and U.S. Pat. No. 4,236,470, the blade portion is formed in neither of the jaws. For this reason, in order to penetrate the tissue, in consideration of extension of the tissue during the puncture, it is necessary to increase a moving distance (an opening and closing stroke) in the direction in which the jaws approach and separate from each other. For this reason, the size of the suture device in the radial direction is exceedingly increased.

On the other hand, according to the endoscope system 1 according to the present embodiment, when the jaw portions 47 and 48 approach each other so that the second puncture member 53 communicates with the first puncture member 54 having the distal end on which the needle member 110 is mounted (the communication state), the tissue can be securely punctured even with a small stroke by the blade portions 53c and 54c.

As described above, the blade portion 53c is formed at the second puncture member 53 throughout substantially the entire circumference of the distal end of the second projecting direction D2. For this reason, the through hole W2 having a size substantially equal to the outside diameter of the second puncture member 53 can be formed at the one edge portion W1, and the whole distal end portion of the second puncture member 53 can pass through the one edge portion W1. Therefore, the length of the jaw portions 47 and 48 in the first projecting direction D1 when the jaw portions 47 and 48 are in the abutting state can be reduced. In comparison with the conventional suture devices, the through hole W2 having a relatively large diameter can be formed in the one edge portion W1, and the wire element 100 can easily pass through the through hole W2.

Since the tissue is sandwiched by the substantially circular blade portion 53c formed at the distal end of the second puncture member 53 in the second projecting direction D2, grasp performance of the tissue is improved. As a result, as the tissue is sandwiched and moved by the present suture device, it is possible to easily determine whether other tissues are involved during the suture.

Even when the second puncture member 53 is reduced in size, since the blade portion 53e does not have a punctiform shape but has a substantially circular shape, stiffness of the blade portion 53c can be maintained.

Furthermore, since the opening of the duct line 53a is formed in the distal end surface of the second puncture member 53, a force applied to the tissue can be concentrated on the blade portion 53c formed at the edge portion of the opening. For this reason, even the through hole having a relatively large diameter can be more easily and securely formed by the blade portion 53c.

The locking member 112 is mounted on a portion of the needle member 110 opposite to the portion thereof connected to the thread main body 120. Therefore, the through hole W2 can be more easily formed in the one edge portion W1 by the locking member 112 disposed at the distal end of the wire element 100.

The suture device 40 includes the opening and closing mechanism, the manipulation wire 65, and the manipulation portion 43. For this reason, as the opening and closing mechanism is manipulated by the manipulation portion 43 via the manipulation wire 65, the operation by which the jaw portions 47 and 48 approach or separate from each other can be easily performed.

The pair of tongue portions 53d is formed at the second puncture member 53. For this reason, the needle member 110 moved in the second puncture member 53 can be securely prevented from moving in the second projecting direction D2.

Since the puncture members 53 and 54 are provided, as the force applied to the one edge portion W1 is further concentrated or a resistance upon penetrating the tissue is suppressed by the distal end portions of the puncture members 53 and 54, the performance of penetrating the tissue can be improved and the through hole W2 can be securely formed.

The second puncture member 53 is formed so as to penetrate the second jaw portion 48, and the duct line 53a is configured to pass through the second puncture member 53. Therefore, the wire element 100 delivered to the second puncture member 53 is removed from the opening of the first projecting direction D1 side of the second puncture member 53, and thus removal of the wire element 100 can be easily performed.

The second jaw portion side slit 48d is formed in the second jaw portion 48. The second puncture member side slit 53b is formed in the second puncture member 53. For this reason, the thread main body 120 is passed through the slits 48d and 53b, and thereby the wire element 100 delivered to the second puncture member 53 can be easily removed from the second jaw portion 48.

The second puncture member 53 and the first puncture member 54 have tubular shapes, respectively. The first puncture member 54 is inserted into the duct line 53a. For this reason, the through hole W2 can be more securely formed by the shearing force applied to the one edge portion W1 by the second puncture member 53 and the first puncture member 54.

The first puncture member side slit 54b is formed in the first puncture member 54. Therefore, as the thread main body 120 is inserted into the first puncture member side slit 54b and is drawn out of the first puncture member 54, the wire element 100 is prevented from easily interfering with the puncture members 53 and 54, and the tissue can be easily sutured.

The thread main body 120 is formed of a material having a larger modulus of longitudinal elasticity than the tissue fixing member 140. For this reason, the outside diameter of the thread main body 120 is set to be smaller than the outside diameter of the tissue fixing member 140. Accordingly, the needle member 110 passing between the edge portions W1 and W3 during suturing can be designed to a small size. Therefore, the puncture members 53 and 54 operated in cooperation with the needle member 110 can also be reduced in size. As a result, the treatment portion 42 or the insertion portion 41 of the suture device 40 can be reduced in size and diameter.

Since the thread main body 120 is formed of a metal, the outside diameter of the thread main body 120 can be further reduced.

Since the tissue fixing member 140 is formed of a resin having biocompatibility, the tissue fixing member 140 can be easily bent along the shape of the sutured edge portions W1 and W3. Furthermore, even when the tissue fixing member 140 is kept in the living body, a burden applied to the living body can be reduced.

Since the outside diameter of the thread main body 120 is smaller than the outside diameter of the tissue fixing member 140, the needle member 110 and further the suture device 40 can be reduced in size.

The intermediate portion 130 and the tissue fixing member 140 are connected by the detachable connecting structure 151. For this reason, the tissue fixing member 140 can be replaced with a new one. Furthermore, for example, as a general suture thread is used as the tissue fixing member 140, the cost of the tissue fixing member 140 can be reduced.

The detachable connecting structure 151 is constituted by the insertion hole 132 of the intermediate portion 130 and the knot 141 of the tissue fixing member 140. For this reason, the detachable connecting structure 151 can be configured simply.

The endoscope system 1 includes the endoscope 10 and the suture system 30. For this reason, the tissue can be sutured by the suture system 30 while observing the inside of the body cavity with the endoscope 10.

The configuration of the endoscope system 1 according to the present embodiment can be variously modified as described below.

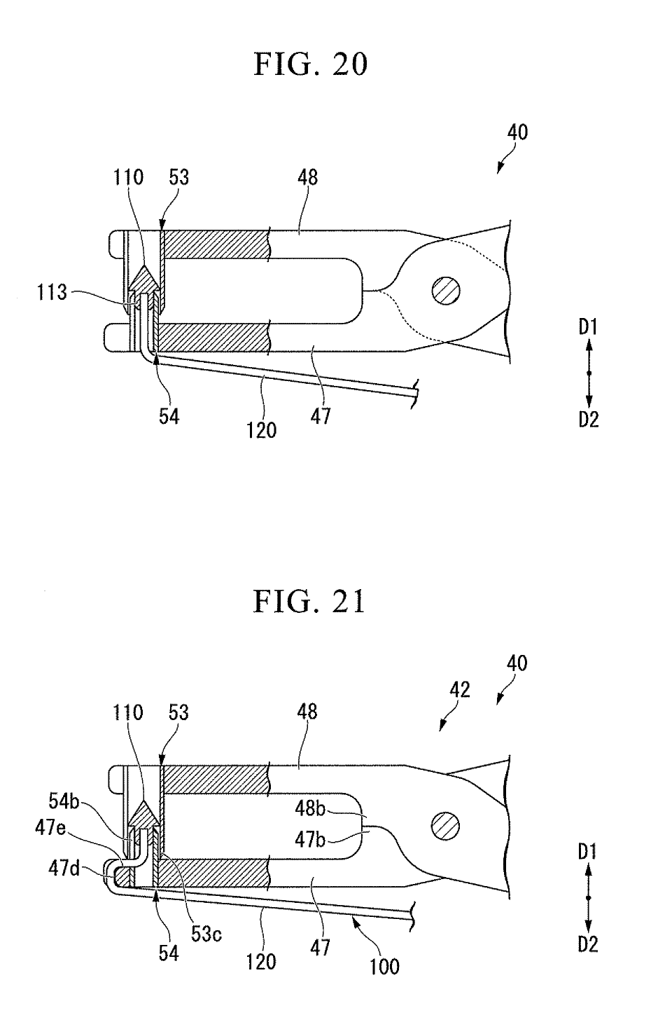

For example, as shown in FIG. 20, the first jaw portion 47 may be configured not to include the bypass slit 47d. In this case, the thread main body 120 is drawn to the proximal side of the suture device 40 through the opening of the second projecting direction D2 side of the duct line 54a of the first puncture member 54. According to the modified example, as the thread main body 120 is pulled to the proximal side, the holding member 113 of the needle member 110 can be securely inserted into the first puncture member 54. In addition, when the needle member 110 is mounted on the first puncture member 54, the thread main body 120 first passes through the first puncture member 54, and thus an axis of the needle member 110 and an axis of the first puncture member 54 can be aligned. For this reason, the needle member 110 can be easily mounted.

As shown in FIG. 21, the length of the second puncture member 53 may be long such that the blade portion 53c comes in contact with the first jaw portion 47 or the gap between the blade portion 53c and the first jaw portion 47 is slightly opened in a state in which the jaw portions 47 and 48 are completely closed. As a result, the sandwiched tissue can be more easily cut. In addition, a second bypass slit 47e communicating with the first puncture member side slit 54b of the first puncture member 54 and the bypass slit 47d of the first jaw portion 47 is formed on a surface of the first projecting direction D1 side of the first jaw portion 47 which is located closer to the distal side than the first puncture member 54. The thread main body 120 is inserted into the first puncture member side slit 54b, the second bypass slit 47e, and the bypass slit 47d, and is drawn from the second projecting direction D2 side of the first jaw portion 47 to the proximal side of the suture device 40. For this reason, even when the jaw portions 47 and 48 are configured such that the distal end of the second puncture member 53 in the second projecting direction D2 abuts the first jaw portion 47 when the jaw portions 47 and 48 are in the abutting state, the thread main body 120 of the wire element 100 is not cut. According to the above-mentioned configuration, the through hole W2 can be more securely formed in the one edge portion W1 by the blade portion 53c of the second puncture member 53 and the first jaw portion 47, which abut each other with no gap therebetween in the abutting state.

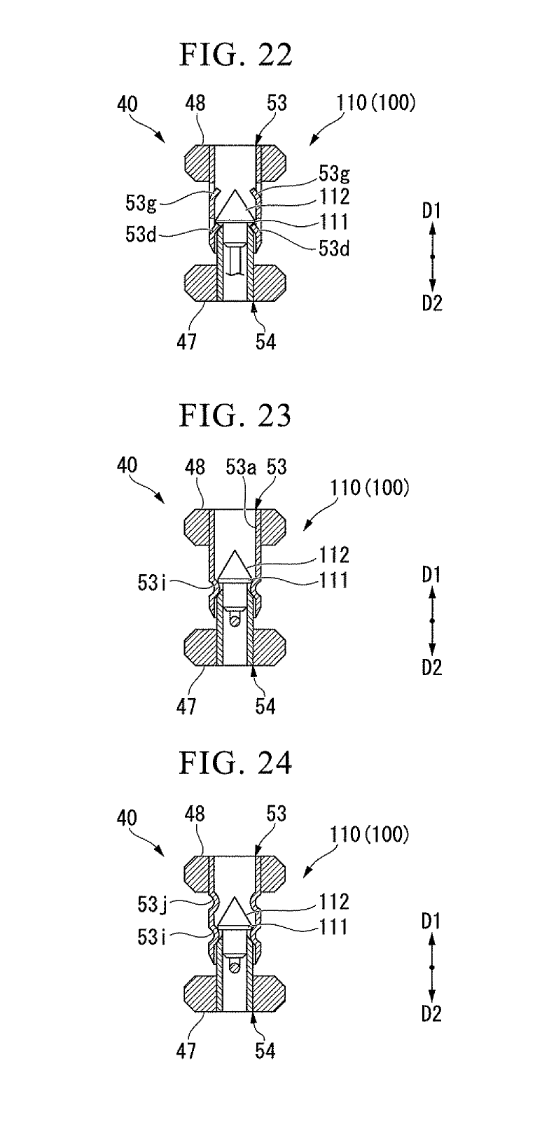

As shown in FIG. 22, in the second puncture member 53, a second tongue portion 53g having substantially the same shape as of the tongue portion 53d may be formed at a position closer to the first projecting direction D1 side of the second puncture member 53 than the tongue portion 53d. According to the above-mentioned configuration, the needle member 110 is moved to the first projecting direction D1 side with respect to the second puncture member 53, and thus the needle member 110 can be prevented from being dropped from the opening of the first projecting direction D1 side of the second puncture member 53.

In the suture device 40 according to the above-mentioned embodiment, the locking portion is the tongue portion 53d. However, as shown in FIG. 23, instead of the pair of tongue portions 53d, a projection (a locking portion) 531 formed so as to project from the entire circumference of the inner circumferential surface of the second puncture member 53 or a part of that toward the duct line 53a side may be provided. For example, the projection 53i is formed by deforming the second puncture member 53 through press processing or partially deforming the second puncture member 53 using a punch, pliers, or the like. As the first puncture member 54 is press-fitted into the projection 53i, the first jaw portion 47 and the second jaw portion 48 are locked.

As the projection 53i is formed at the second puncture member 53 instead of the pair of tongue portions 53d, cutout of the second puncture member 53 is not needed. For this reason, processing of the second puncture member 53 can be easily performed. In addition, the stiffness of the projection 531 is greater than that of the tongue portion 53d. For this reason, a force applied to the press-fitted first puncture member 54 can be increased. Further, repetition durability of the second puncture member 53 can be increased.

In the modified example, as shown in FIG. 24, in the second puncture member 53, a second projection 53j having substantially the same shape as the projection 53i may be formed at a position closer to the first projecting direction D1 side of the second puncture member 53 than the projection 53i. According to the above-mentioned configuration, the needle member 110 is moved to the first projecting direction D1 side, and thus the needle member 110 can be prevented from being dropped from the opening of the first projecting direction D1 side of the second puncture member 53.

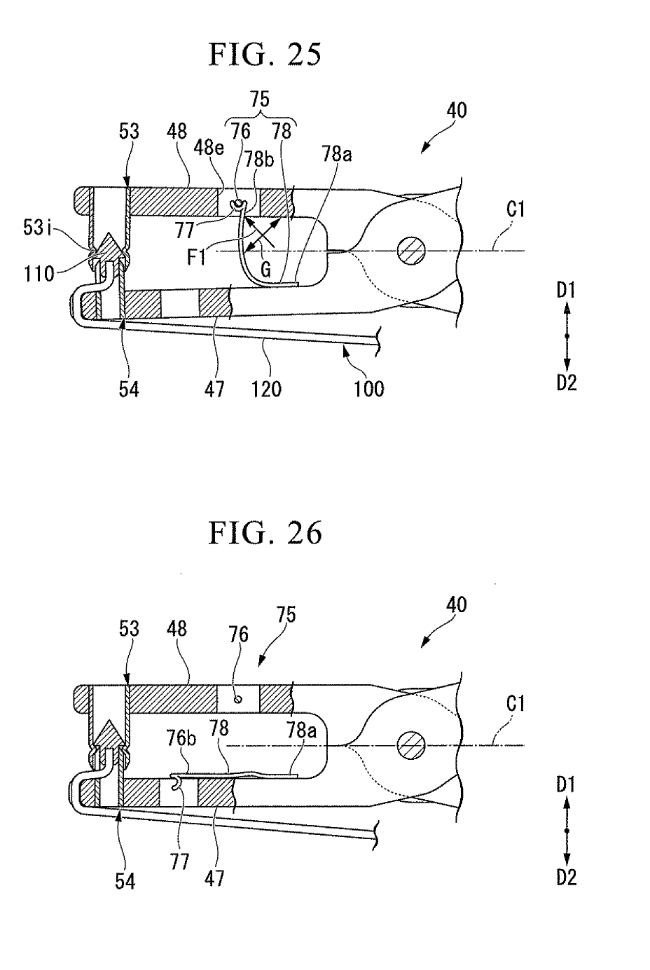

Like the suture device 40 shown in FIG. 25, in a state in which the needle member 110 does not engage with the second puncture member 53 (hereinafter referred to as “a pre-abutting state”), a locking motion preventing mechanism 75 configured to hold the first jaw portion 47 and the second jaw portion 48 such that they do not approach each other may be provided.

The locking motion preventing mechanism 75 has a pin (an engaging member) 76 mounted on the second jaw portion 48, and a shaft-shaped member 78. A proximal end 78a of the shaft-shaped member 78 is connected to the first jaw portion 47. A concave portion (an engaged member) 77 engaging with the pin 76 is formed at a distal end 78b of the shaft-shaped member 78. In the modified example, the concave portion 77 and the shaft-shaped member 78 are integrally formed by a resilient member such as a flat spring or the like.

The pin 76 is mounted in an attachment hole 48e formed in the second jaw portion 48 so as to extend in a direction perpendicular to both the axis C1 of the base member 45 and the first projecting direction D1.

As shown in FIG. 26, the shaft-shaped member 78 is disposed to extend substantially parallel to the axis C1 in a natural state in which the external force is not applied.

As shown in FIG. 25, when the locking motion preventing mechanism 75 is used, the shaft-shaped member 78 is elastically deformed about the proximal end 78a so as to be curved in the plate thickness direction, and the concave portion 77 is engaged with the pin 76. Here, the concave portion 77 is configured to engage with the pin 76 in both an opposed direction F1 directed from the proximal end 78a of the shaft-shaped member 78 to the pin 76, and a perpendicular direction G which is substantially perpendicular to the opposed direction F1 and is on a plane surface on which the elastically deformed shaft-shaped member 78 is disposed.

When the concave portion 77 engages with the pin 76, the positions of the jaw portions 47 and 48 are in the above-mentioned pre-abutting state, and the needle member 110 is not locked by the second puncture member 53. Furthermore, since the concave portion 77 engages with the pin 76 in the perpendicular direction G, in this state, the concave portion 77 is not separated from the pin 76.

The insertion portion 41 of the suture device 40 is inserted through the forceps channel 11 of the endoscope 10 while a force is applied to the wire slider 69 in a direction in which the wire slider 69 is slid to the proximal side, such that the jaw portions 47 and 48 approach each other after the jaw portions 47 and 48 reach the pre-abutting state using the locking motion preventing mechanism 75.

When the treatment portion 42 is used, the treatment portion 42 projects forward from the forceps channel 11, and the wire slider 69 is slid to the distal side to separate the jaw portions 47 and 48 from each other. Then, as the shaft-shaped member 78 returns to the shape shown in FIG. 26 by its own elastic force, engagement of the pin 76 and the concave portion 77 is released. After that, as the wire slider 69 is slid to the proximal side, the jaw portions 47 and 48 can be in the abutting state, and the needle member 110 can be moved to the second puncture member 53.

Since the locking motion preventing mechanism 75 is provided as described above, the jaw portions 47 and 48 can be prevented from being locked by the locking unit by mistake.

In the modified example, while the pin 76 is mounted on the second jaw portion 48 and the shaft-shaped member 78 is mounted on the first jaw portion 47, the shaft-shaped member 78 may be mounted on the second jaw portion 48 and the pin 76 may be mounted on the first jaw portion 47.

In the modified example, while the shaft-shaped member 78 is constituted by a flat spring, the shaft-shaped member 78 may be constituted by another element. For example, the shaft-shaped member 78 may also be constituted by a hinge joint and a torsion spring configured to bias the hinge joint in a rotational direction.

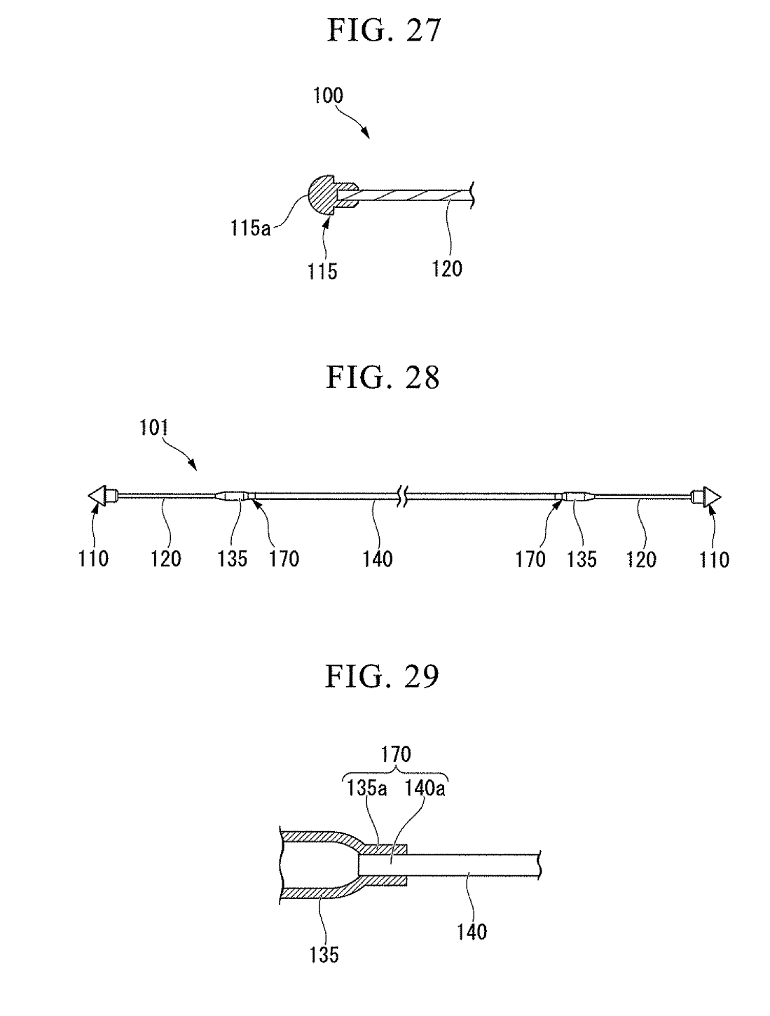

Like the wire element 100 shown in FIG. 27, a locking member 115a formed at a portion of a needle member 115 opposite to a portion thereof connected to the thread main body 120 may have a substantially hemispherical shape.

The suture device may be used to stop the bleeding from the blood vessel in addition to suturing of the opening. In this case, the blood vessel is tied by the wire element from the outer circumferential surface side to stop the bleeding. As the locking member 115a is formed as described in the modified example, when the needle member 115 comes in contact with the tissue or the needle member 115 is dropped by mistake, invasion to the surrounding tissue can be reduced.

The wire element 101 may be configured as shown in FIG. 28. The wire element 101 is configured to be symmetrical with respect to the tissue fixing member 140 arranged at a center.

An intermediate portion 135 is connected to the first end of the tissue fixing member 140, and the above-mentioned thread main body 120 is connected to a side of the intermediate portion 135 opposite to a side of the intermediate portion 135 connected to the fixing member 140.

In the modified example, as shown in FIG. 29, the intermediate portion 135 and the tissue fixing member 140 are connected by a fixing connecting structure 170, in which the intermediate portion 135 and the tissue fixing member 140 are fixed with each other and integrated. The fixing connecting structure 170 is formed of a first end 140a of the tissue fixing member 140 and the intermediate portion 135. The fixing connecting structure 170 is constituted by a connecting concave portion 135a into which the first end 140a of the tissue fixing member 140 is inserted and by which the first end 140a is fastened such that side surfaces of the first end 140a is sandwiched. For example, the connecting concave portion 135a can be formed by deforming (caulking) to crush a tubular member formed of metal or the like.

For example, the wire element 101 can be mounted on the above-mentioned suture device 40 and used. Here, the wire element 101 is used with two suture devices 40. Even when it is difficult for the operator to mount the needle member 110 on the first puncture member 54, the suture system is sold in a state in which the needle members 110 of the wire element 101 are mounted on the two suture devices 40, respectively. The suture device 40 may be a disposable type.

The operator inserts each of the insertion portions 41 of the purchased suture system into the forceps channel 11 of the endoscope 10. Then, the edge portions W1 and W3 are sutured by one stitch each with the suture devices 40, for a total of two stitches.

As the wire element 101 having the above-mentioned configuration includes the fixing connecting structure 170, the intermediate portion 135 and the tissue fixing member 140 can be more securely connected. Furthermore, the fixing connecting structure 170 can be configured by a simple configuration, i.e., the first end 140a of the tissue fixing member 140 and the connecting concave portion 135a.

In addition, since the operator need not mount the needle member 110 on the first puncture member 54 in the suture system having the above-mentioned configuration, time necessary for the procedure can be reduced. Since each of the needle members 110 merely sutures by one stitch, a decrease in performance of puncture of the needle member 110 can be prevented.

### Second Embodiment

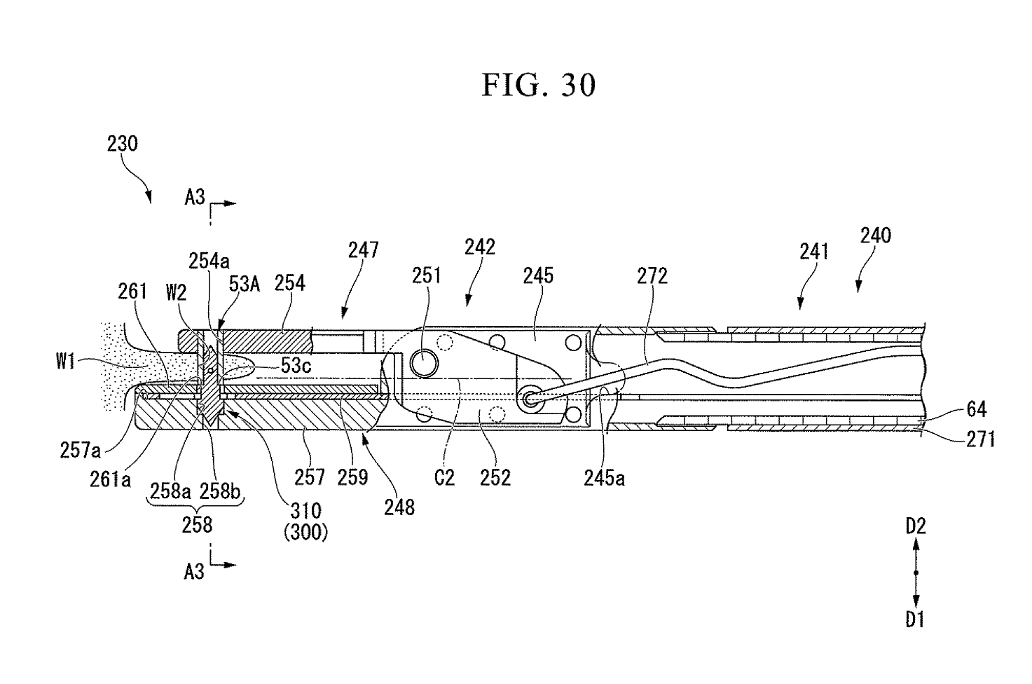

Next, an endoscope system according to a second embodiment of the present invention is described referring to FIGS. 30 to 35. However, the same elements in the first embodiment are designated by the same reference numerals and a description thereof is omitted here, and the second embodiment is described focusing on differences therefrom.

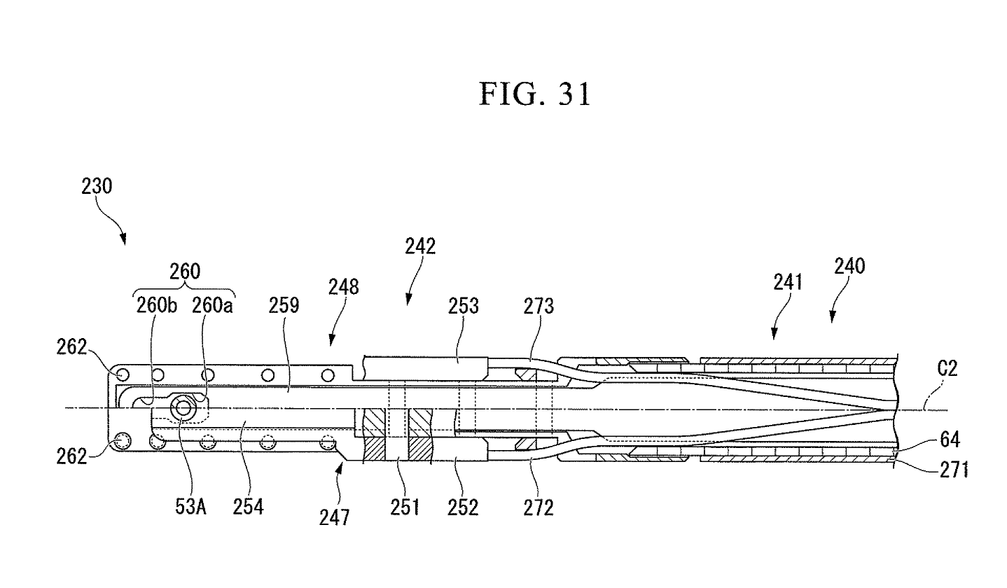

The endoscope system according to the second embodiment of the present invention is different from the first embodiment in terms of only the endoscope system and the suture system. As shown in FIGS. 30 and 31, a suture system 230 includes a suture device 240, and a wire element 300 that is detachably mounted on the suture device 240.

The suture device 240 includes a long insertion portion 241, a treatment portion 242 installed at a distal end of the insertion portion 241, and a manipulation portion 243 (see FIG. 33) that is provided at a proximal end of the insertion portion 241 and is configured to manipulate the treatment portion 242. Hereinafter, first, the treatment portion 242 is described.

The treatment portion 242 has a base member 245, a first jaw portion 247 rotatably connected to the base member 245, and a second jaw portion 248 fixed to the base member 245. The jaw portions 247 and 248 are formed so as to extend in an axis C2 direction of the treatment portion 242.

A notch 245a into which manipulation wires 272 and 273 (to be described below) are inserted is formed at the base member 245.

The first jaw portion 247 includes arms 252 and 253 supported by a hinge pin 251 fixed to the base member 245 so as to rotate about the hinge pin 251, and a jaw portion main body 254. The jaw portion main body 254 is fixed between the arm 252 and the arm 253. The proximal sides of the arms 252 and 253 are disposed such that the base member 245 is sandwiched between the proximal sides of the arms 252 and 253. The hinge pin 251 is fixed to a central part of the arms 252 and 253 in the axis C2 direction.

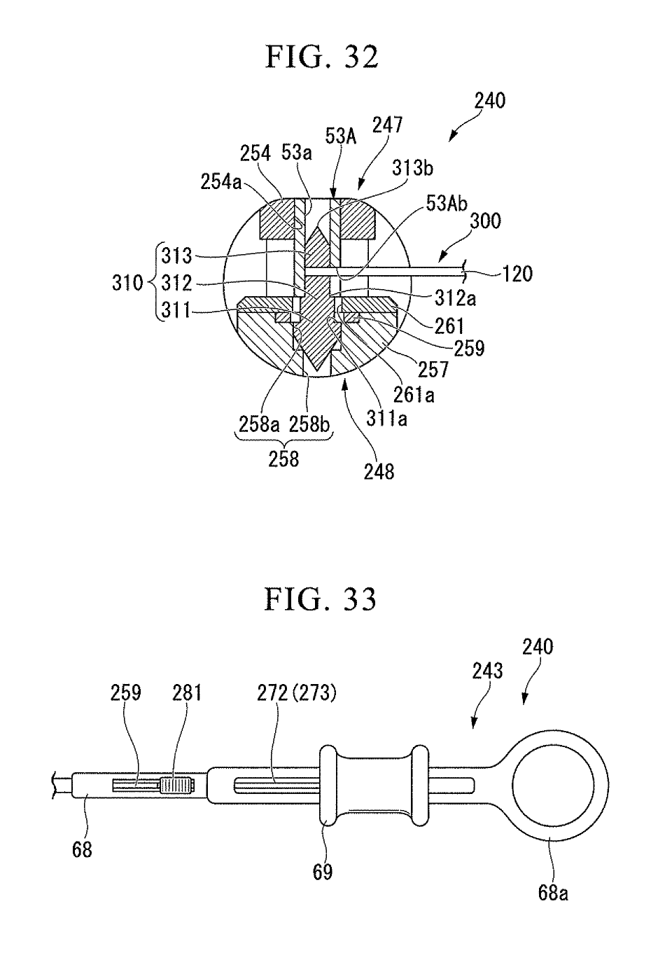

As shown in FIGS. 30 and 32, a fixing hole 254a penetrating the jaw portion main body 254 in the first projecting direction D1 is formed in the distal side of the jaw portion main body 254. A first puncture member 53A having the same shape as the above-mentioned second puncture member 53 is fixed to the fixing hole 254a. In the first puncture member 53A of the present embodiment, the pair of tongue portions 53d is not formed. A first puncture member side slit 53Ab is formed in a direction perpendicular to both the axis C2 direction of the first puncture member 53A and the first projecting direction D1 (see FIG. 32).

The second jaw portion 248 has a jaw portion main body 257 formed in a flat plate shape. A proximal end of the jaw portion main body 257 is fixed to the base member 245. A groove portion 257a extending in the axis C2 direction is formed in a surface of the second projecting direction D2 side of the jaw portion main body 257. A support hole (a second internal space) 258 extending in the second projecting direction D2 and penetrating the jaw portion main body 257 is formed in a bottom surface of the distal side of the groove portion 257a. The support hole 258 is constituted by a support hole large diameter portion 258a formed at the second projecting direction D2 side, and a support hole small diameter portion 258b formed at the first projecting direction D1 side. An inside diameter of the support hole small diameter portion 258b is smaller than that of the support hole large diameter portion 258a. The support hole large diameter portion 258a and the support hole small diameter portion 258b are concentrically formed.

A sheet-shaped lock switching plate (a locking portion) 259 is mounted in the groove portion 257a so as to slide in the axis C2 direction. For example, the lock switching plate 259 may be formed by a plate-shaped member formed of a metal. The thickness of the lock switching plate 259 is set to a value smaller than a depth of the groove portion 257a. As shown in FIG. 31, a locking hole 260 penetrating the lock switching plate 259 in the second projecting direction D2 is formed in the distal side of the lock switching plate 259. The locking hole 260 is constituted by a locking hole large diameter portion 260a formed at the proximal side of the locking hole 260, and a locking hole small diameter portion 260b formed at the distal side of the locking hole 260. The locking hole small diameter portion 260b is set to have a width smaller than that of the locking hole large diameter portion 260a. The locking hole large diameter portion 260a communicates with the locking hole small diameter portion 260b in the axis C2 direction. The width of the locking hole large diameter portion 260a is set to be substantially equal to the inside diameter of the support hole large diameter portion 258a.

As shown in FIGS. 30 and 32, a plate-shaped switching plate holding member 261 is mounted on a surface of the second projecting direction D2 side of the jaw portion main body 257. A through hole 261a having a size such that the through hole 261a overlaps the support hole large diameter portion 258a of the jaw portion main body 257 when seen from a plan view is formed in the switching plate holding member 261.

The jaw portion main body 257 and the switching plate holding member 261 are connected by brazing, caulking, or the like, using a plurality of pins 262 extending in the second projecting direction D2.

In the treatment portion 242 having the above-mentioned configuration, the lock switching plate 259 can be slid in the axis C2 direction with respect to the jaw portion main body 257. The position of the lock switching plate 259 in the axis C2 direction is adjusted by the manipulation portion 243. A locking switch mechanism is constituted by the lock switching plate 259 and the manipulation portion 243.

In a moved state in which the lock switching plate 259 is slid to the distal side to a maximum extent, the through hole 261a of the switching plate holding member 261 overlaps the locking hole large diameter portion 260a of the lock switching plate 259 when seen in a plan view. On the other hand, in a locked state in which the lock switching plate 259 is slid to the proximal side to a maximum extent, the through hole 261a of the switching plate holding member 261 overlaps the locking hole small diameter portion 260b of the lock switching plate 259 when seen in a plan view.

When the jaw portions 247 and 248 approach each other and a distal end of the first puncture member 53A in the first projecting direction D1 abuts the switching plate holding member 261, the jaw portions 247 and 248 reach the abutting state. Here, the first puncture member 53A is disposed on the same axis as the through hole 261a of the switching plate holding member 261 to be, and the first puncture member 53A and the support hole 258 reach a communication state in which the duct line 53a of the first puncture member 53A communicates with the duct line of the support hole 258.

In order to improve assembling work efficiency, the jaw portion main body 257 may be divided into two parts in the width direction, and the two parts may be connected by a pin.

The insertion portion 241 includes a long outer sheath 271 extending in the axis C2 direction, the above-mentioned inner sheath 64 inserted into the outer sheath 271, and a first manipulation wire (a manipulation wire) 272 and a second manipulation wire (a manipulation wire) 273 inserted into the inner sheath 64.

In the suture device 240 according to the present embodiment, a single-layer coil is used as the outer sheath 271.

Curved shapes, which does not change in shape during manipulating the manipulation wires 272 and 273, are formed at the distal sides of the manipulation wires 272 and 273.

The first manipulation wire 272 has a distal end connected to a proximal end of the arm 252 via the notch 245a. Similar to the second manipulation wire 273, a distal end of the second manipulation wire 273 is connected to a proximal end of the arm 253.

The above-mentioned lock switching plate 259 is inserted into the inner sheath 64 and extends to the proximal side.

As shown in FIG. 33, the manipulation portion 243 of the suture device 240 according to the present embodiment includes a switching plate slider 281 that can slide with respect to the manipulation portion main body 68, in addition to the configurations of the manipulation portion 43 of the suture device 40 according to the first embodiment.

A proximal end of the lock switching plate 259 is connected to the switching plate slider 281. Proximal ends of the manipulation wires 272 and 273 are connected to the wire slider 69.

A ratchet mechanism (not shown) is provided at the wire slider 69 and the switching plate slider 281. The ratchet mechanism can hold the positions of the sliders 69 and 281 with respect to the manipulation portion main body 68 or release the holding.

In the suture device 240 having the above-mentioned configuration, as the wire slider 69 is slid with respect to the manipulation portion main body 68, the first jaw portion 247 can rotate about the hinge pin 251. Since the curved shapes are formed at the distal sides of the manipulation wires 272 and 273, the first jaw portion 247 can smoothly rotate about the hinge pin 251. In addition, as the switching plate slider 281 is slid, the position of the lock switching plate 259 can be switched between the position in the moved state and the position in the locked state.

As shown in FIGS. 30 and 32, the wire element 300 includes a needle member 310 instead of the needle member 110 of the wire element 100 according to the first embodiment.

The needle member 310 has a locking member 311 having a substantially conical shape, a first holding member 312 having a smaller diameter than the locking member 311, and a second holding member 313 having a smaller diameter than the first holding member 312. The first holding member 312 and the second holding member 313 correspond to the holding member.

The first holding member 312 is arranged adjacent to the locking member 311 in the axial direction and is connected to the locking member 311. The second holding member 313 is arranged adjacent to the first holding member 312 in the axial direction at an opposite side of the locking member 311 and is connected to the first holding member 312. The locking member 311, the first holding member 312 and the second holding member 313 are concentrically disposed. A first stepped portion 311a is formed by a difference in diameter between the locking member 311 and the first holding member 312. A second stepped portion 312a is formed by a difference in diameter between the first holding member 312 and the second holding member 313.

A needle-shaped body 313b having a diameter that reduces as it moves away from the first holding member 312 is formed at an opposite side of the first holding member 312 in the second holding member 313.

The locking member 311, the first holding member 312 and the second holding member 313 are integrally formed of a metal such as stainless steel, titanium, a titanium alloy, or the like.

A first end of the thread main body 120 is connected to the second holding member 313.

The outside diameter of the locking member 311 is set to be smaller than the inside diameter of the support hole large diameter portion 258a, the width of the locking hole large diameter portion 260a, and the inside diameter of the through hole 261a. In addition, the outside diameter of the locking member 311 is set to be larger than the inside diameter of the support hole small diameter portion 258b and the width of the locking hole small diameter portion 260b.

The outside diameter of the second holding member 313 is set to be slightly smaller than the inside diameter of the duct line 53a of the first puncture member 53A. The outside diameter of the second holding member 313 is set such that an appropriate frictional force is generated between the inner circumferential surface of the duct line 53a and the second holding member 313 when the second holding member 313 mounted on the duct line 53a of the first puncture member 53A is removed from the first puncture member 53A. For this reason, the needle member 310 is configured not to be easily removed from the first puncture member 53A. When the second holding member 313 is mounted on the first puncture member 53A, the second stepped portion 312a of the needle member 310 is locked by the distal end of the first puncture member 53A in the first projecting direction D1. In addition, the needle-shaped body 313b is formed at the needle member 310. For this reason, the needle-shaped body 313b serves as a guide upon insertion, and thus the second holding member 313 can be easily mounted on the first puncture member 53A.

Next, the procedure using the endoscope system according to the present embodiment is described. The lock switching plate 259 is set to the moved state when the procedure starts.

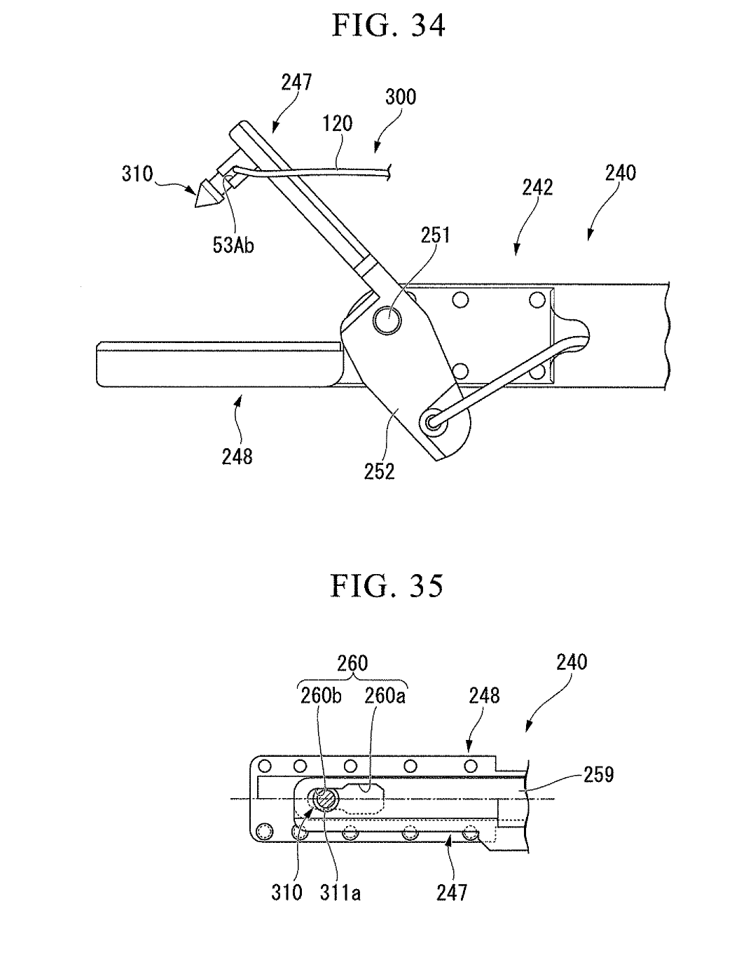

The operator slides the wire slider 69 to the distal side with respect to the manipulation portion main body 68 at the outside of the body of the patient, and as shown in FIG. 34, separates the jaw portions 247 and 248 from each other (an open state). As the second holding member 313 of the needle member 310 is inserted into the first puncture member 53A, the wire element 300 is mounted on the first puncture member 53A of the suture device 240. Here, the thread main body 120 is inserted into the first puncture member side slit 53Ab and is drawn to the proximal side of the suture device 240. Then, the wire slider 69 is slid to the proximal side, and as shown in FIG. 30, the jaw portions 247 and 248 abut each other (the abutting state).

Next, the insertion portion 241 of the suture device 240 is inserted into the forceps channel 11 of the endoscope 10, and the suture device 40 is mounted on the endoscope 10.

When the distal end of the endoscope insertion portion 12 arrives at a position opposite to the opening formed in the stomach wall, the treatment portion 242 projects forward from the forceps channel 11. The jaw portions 247 and 248 are opened, and the one edge portion W1 of the opening is disposed between the jaw portions 247 and 248. As the jaw portions 247 and 248 reach the abutting state again, as shown in FIG. 30, the through hole W2 is formed in the one edge portion W1 by the blade portion 53e of the first puncture member 53A and the switching plate holding member 261, and further the locking member 311 of the needle member 310.

When the switching plate slider 281 is slid to the proximal side so that the lock switching plate 259 is in the locked state, as shown in FIG. 35, the locking hole small diameter portion 260b of the lock switching plate 259 engages with the first stepped portion 311a of the needle member 310. For this reason, the needle member 310 cannot move to the second projecting direction D2 side with respect to the second jaw portion 248. As described above, as the moved state and the locked state are switched by a locking switch mechanism constituted by the lock switching plate 259 and the manipulation portion 243, engagement or disengagement of the lock switching plate 259 with the needle member 310 is switched.

Here, when the jaw portions 247 and 248 are opened, as a force occurred by engaging the locking hole small diameter portion 260b with the first stepped portion 311a overcomes the frictional force between the second holding member 313 and the duct line 53a, the needle member 310 is removed from the first puncture member 53A. For this reason, the needle member 310 is mounted in the support hole 258.

As a portion of the insertion portion 241 of the suture device 240 is retracted to the forceps channel 11 or the entire endoscope 10 is moved, the tissue fixing member 140 is inserted into the through hole W2.

When the jaw portions 247 and 248 reach the abutting state at a place separated from the one edge portion W1 in the body, the second holding member 313 of the needle member 310 is inserted into the first puncture member 53A. When the lock switching plate 259 reaches the moved state as the switching plate slider 281 is slid to the distal side, engagement between the lock switching plate 259 and the needle member 310 is released. The needle member 310 is moved to the second projecting direction D2 side with respect to the second jaw portion 248. When the jaw portions 247 and 248 are opened again, the jaw portions 247 and 248 are separated from each other in a state in which the wire element 300 is mounted on the first puncture member 53A.

Next, the other edge portion W3 of the opening W is sutured by one stitch, and then the needle member 310 is delivered from the second jaw portion 248 to the first jaw portion 247.

The above-mentioned procedure is continuously performed in the body. As the edge portions W1 and W3 are sequentially sutured, the continuous suture can be performed.

As described above, according to the endoscope system of the present embodiment, the wire element 300 is changed from the state in which the wire element 300 is mounted on the first puncture member 53A to the state in which the wire element 300 is mounted in the support hole 258. Therefore, when the jaw portions 247 and 248 are separated from each other to be opened and the through hole W2 is disposed at a central part between the jaw portions 247 and 248, only the wire element 300 can be inserted into the through hole W2 of the one edge portion W1.

In the suture system 230 according to the present embodiment, after the needle member 310 is delivered from the first jaw portion 247 to the second jaw portion 248, the needle member 310 is delivered from the second jaw portion 248 to the first jaw portion 247 at a place separated from the opening. However, even when the needle member 310 is delivered from the second jaw portion 248 to the first jaw portion 247, the edge portions W1 and W3 may be sutured.

### Third Embodiment

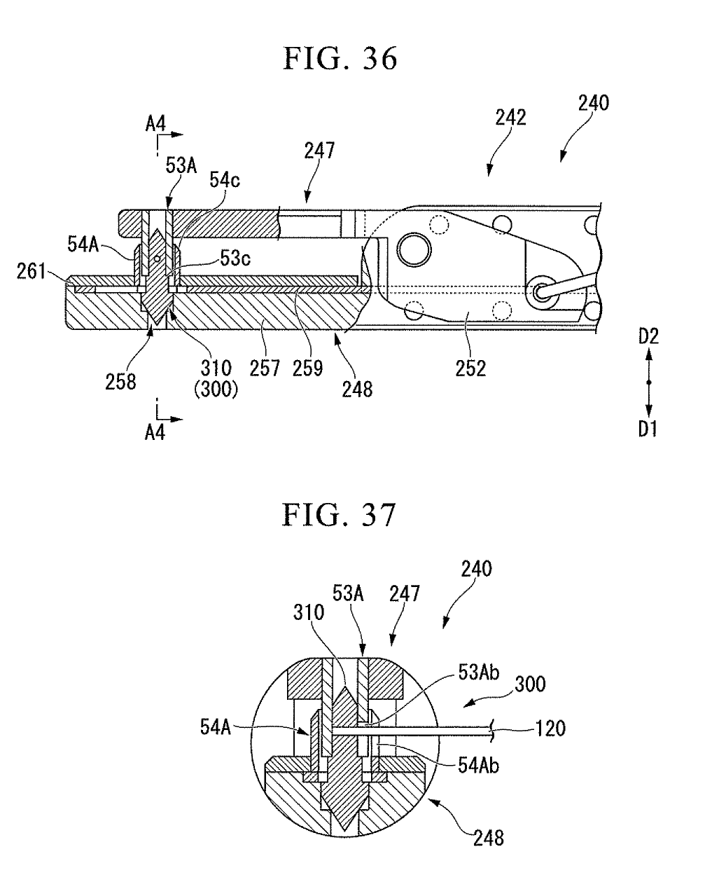

In addition, in the endoscope system according to the second embodiment of the present invention, as shown in FIG. 36, the second puncture member 54A having the same shape as the above-mentioned first puncture member 54 projecting to the second projecting direction D2 side may be provided at the switching plate holding member 261. A modified example of the endoscope system according to the second embodiment is a third embodiment of the present invention. The endoscope system according to the third embodiment of the present invention is described referring to FIGS. 36 and 37.

Brazing, laser welding, or the like, can be used as a method of fixing a second puncture member 54A to the switching plate holding member 261.

In the suture device 240 according to the third embodiment, when the jaw portions 247 and 248 are in the abutting state, the first puncture member 53A is configured to be inserted into the second puncture member 54A.

As shown in FIG. 37, when the jaw portions 247 and 248 are in the abutting state, the thread main body 120 is inserted into the first puncture member side slit 53Ab of the first puncture member 53A and a second puncture member side slit 54Ab of and the second puncture member 54A and is drawn to the proximal side of the suture device 240.

As the suture device 240 has the above-mentioned configuration, the through hole W2 can be more securely and easily formed by a shearing force between the blade portion 53c of the first puncture member 53A and the blade portion 54c of the second puncture member 54A.

Hereinabove, while preferred embodiments of the present invention have been described, the present invention is not limited to the embodiments. Additions, omissions, substitutions, combinations, and other modifications may be made to the present invention without departing from the spirit and scope of the present invention. The present invention is not limited to the above-mentioned description, and is only limited by the appended claims.

For example, the detachable connecting structure of the wire element according to the above-mentioned embodiment is constituted by the insertion hole 132 of the intermediate portion 130 and the knot 141 of the tissue fixing member 140. However, the detachable connecting structure is not limited thereto, but the intermediate portion 130 and the tissue fixing member 140 may be releasably connected by a hook-and-loop fastener or a clip.

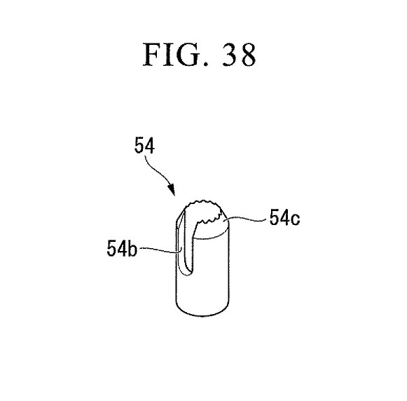

As shown in FIG. 38, a distal end shape of the blade portion 54c may be formed such that a wave form is formed in the circumferential direction. According to the above-mentioned configuration, the tissue is easily cut by the blade portion 54c, and the through hole W2 can be more securely formed.

In the endoscope system according to the above-mentioned embodiment, the treatment system is the suture system configured to suture the tissue. However, in the above-mentioned suture system, the treatment system may form a hole in the tissue without including the wire element.

In the wire element according to the above-mentioned embodiment, while the needle member 110 is fixed to the thread main body 120, the needle member 110 may be fixed to the thread main body 120 only in a longitudinal axial direction of the thread main body 120.

### BRIEF DESCRIPTION OF THE REFERENCE SYMBOLS

1: endoscope system
10: endoscope
11: forceps channel (channel)
12: endoscope insertion portion
13: endoscope manipulation portion
14: distal end rigid portion
15: bending portion
16: flexible tube portion
19: lighting unit
20: observation unit
21: manipulation dial
22: universal cable
23: forceps stopper
30, 230: suture system
40, 240: suture device
41, 241: insertion portion
42, 242: treatment portion
43, 243: manipulation portion
45, 245: base member (supporting portion)
47, 247: first jaw portion (first grasping member)
47a, 48a: through hole
47b: first abutting member
47c, 48c: fixing hole
47d: bypass slit
47e: second bypass slit
48, 248: second jaw portion (second grasping member)
48b: second abutting member
48d: second jaw portion side slit
48e: attachment hole
49, 50: coupling member
52, 251: hinge pin
53, 54A: second puncture member
53a: duct line
53b, 54Ab: second puncture member side slit
53c: blade portion
53d: pair of tongue portions (locking portion)
53f: end face
53g: second tongue portion
53i: projection (locking portion)
53j: second projection
54, 53A: first puncture member
54a: duct line (internal space)
54b, 53Ab: first puncture member side slit (first slit)
54c: blade portion (abutting portion)
57, 58: coupling pin
59: connecting member
60: hinge pin
63, 271: outer sheath
64: inner sheath
65, 272, 273: manipulation wire (manipulation member)
65a: stainless steel pipe
68: manipulation portion main body
68a: finger hooking ring
69: wire slider
69a: depression
75: locking motion preventing mechanism
76: pin (engaging member)
77: concave portion (engaged member)
78: shaft-shaped member
78a: proximal end
78b: distal end
100, 101, 300: wire element
110, 115, 310: needle member
112, 115a, 311: locking member
113: holding member
120: thread main body
130, 135: intermediate portion
131: end portion
132: insertion hole
135a: connecting concave portion
140: tissue fixing member
141: knot
151: detachable connecting structure
152: bar
160: fixing mechanism
161: main body
161a, 161b, 162a: through hole
162: lock member
162b: slope portion
163: sealing member
170: fixing connecting structure
245a: notch
252, 253: arm
254: jaw portion main body
254a: fixing hole
257: jaw portion main body
257a: groove portion
258: support hole (second internal space)
258a: support hole large diameter portion
258b: support hole small diameter portion
259: lock switching plate (locking portion)
260: locking hole
260a: locking hole large diameter portion
260b: locking hole small diameter portion
261: switching plate holding member
261a: through hole
262: pin
281: switching plate slider
311a: first stepped portion
312: first holding member
312a: second stepped portion
313: second holding member
313b: needle-shaped body

What is claimed is:

1. A treatment system comprising:
a supporting portion provided so as to extend from a distal side to a proximal side thereof;
a first grasping member and a second grasping member which are supported by the supporting portion so as to approach and separate from each other, and grasp a tissue;
a needle member including a distal end portion which has a sharp distal end and punctures the tissue, and a proximal end portion which is continuous with the distal end portion;
a holding portion which is mounted on the first grasping member, releasably holds the proximal end portion of the needle member, and holds the proximal end portion of the needle member such that the distal end portion of the needle member is exposed to an outside when the first grasping member and the second grasping member are separated from each other; and
a locking portion which is mounted on the second grasping member such that the locking portion locks the distal end portion of the needle member at a position at which the first grasping member and the second grasping member approach each other, and locks the distal end portion of the needle member such that the proximal end portion of the needle member is removed from the holding portion when the first grasping member and the second grasping member are separated from each other from the position at which the first grasping member and the second grasping member approach each other.

2. The treatment system according to claim 1, further comprising:
a manipulation member which is connected to both the first grasping member and the second grasping member, and freely moves in an extending direction of the supporting portion;
a manipulation portion allowing the first grasping member and the second grasping member to approach and separate from each other by the manipulation portion moving the manipulation member;
a first puncture member formed so as to project from the first grasping member toward the second grasping member;
an abutting portion provided at a distal end portion of the first puncture member;
a second puncture member which is arranged at a position opposite to the first puncture member, and is formed so as to project from the second grasping member toward the first grasping member;
a blade portion provided at a distal end portion of the second puncture member; and
an opening and closing mechanism allowing the blade portion and the abutting portion to be in an abutting state in which the blade portion abuts the abutting portion by the opening and closing mechanism allowing the first grasping member and the second grasping member to approach each other from a position at which the first grasping member and the second grasping member are separated from each other.

3. The treatment system according to claim 2, further comprising
a wire member having flexibility, wherein the proximal end portion of the needle member is mounted on one end of the wire member.

4. The treatment system according to claim 3, wherein
a first internal space is formed in the first puncture member so as to communicate with a first opening formed at a distal end of the first puncture member in a first projecting direction in which the first puncture member projects,
a first slit which communicates with the first internal space and into which the wire member is inserted is formed in at least the distal end portion of the first puncture member in the first projecting direction,
a second internal space is formed in the second puncture member so as to communicate with a second opening formed in a distal end of the second puncture member in a second projecting direction in which the second puncture member projects, and
a second slit which communicates with the second internal space and into which the wire member is inserted is formed in both the second grasping member and the second puncture member throughout an entire length in the second projecting direction.

5. The treatment system according to claim 4, further comprising:
an engaging member mounted on one of the first grasping member and the second grasping member; and
a shaft-shaped member having elasticity, wherein a proximal end of the shaft-shaped member is connected to an other of the first grasping member and the second grasping member, and a engaged member which is engaged with the engaging member is mounted on a distal end of the shaft-shaped member,
wherein when the shaft-shaped member is elastically deformed about the proximal end of the shaft-shaped member and the engaged member engages with the engaging member, the engaged member engages with the engaging member in both an opposed direction from the proximal end of the shaft-shaped member toward the engaging member and a direction which is substantially perpendicular to the opposed direction and is on a plane surface on which the shaft-shaped member elastically deformed is disposed, and
the first grasping member and the second grasping member are separated from each other from the abutting state when the engaged member of the shaft-shaped member elastically deformed engages with the engaging member.

6. The treatment system according to claim 5, wherein
the holding portion is formed at an inner wall surface which forms the first internal space, and
the holding portion generates the holding force by a frictional force generated between the needle member and the holding portion.

7. The treatment system according to claim 6, wherein
the first puncture member and the second puncture member have tubular shapes, and
one of the first puncture member and the second puncture member is inserted into an other of the first puncture member and the second puncture member.

8. The treatment system according to claim 7, wherein
the needle member includes a holding member capable of being held by the holding portion, and a locking member capable of being locked by the locking portion,
the holding member is connected to the locking member and has an outside diameter smaller than an outer diameter of the locking member, and
the wire member includes:
a thread main body, wherein one end of the thread main body is connected to the holding member, and an outside diameter of the thread main body is set to be smaller than the outside diameter of the locking member;
an intermediate portion connected to an other end of the thread main body; and
a tissue fixing member disposed at an opposite side of the thread main body, wherein one end of the tissue fixing member is connected to the intermediate portion.

9. The treatment system according to claim 8, wherein
the thread main body is formed of a material having a larger modulus of longitudinal elasticity than the tissue fixing member.

10. The treatment system according to claim 9, wherein
the thread main body is formed of a metal.

11. The treatment system according to claim 9, wherein
the tissue fixing member is formed of a resin having biocompatibility.

12. The treatment system according to claim 8, wherein
the outside diameter of the thread main body is smaller than an outside diameter of the tissue fixing member.

13. The treatment system according to claim 8, wherein
the locking member has a substantially hemispherical shape.

14. The treatment system according to claim 8, wherein
the intermediate portion and the tissue fixing member are detachably connected with each other by a detachable connecting structure.

15. The treatment system according to claim 14, wherein
the detachable connecting structure includes:
an insertion hole formed in the intermediate portion; and
a knot which is formed at the tissue fixing member, is inserted into the insertion hole, and is tied to the intermediate portion.

16. The treatment system according to claim 8, wherein
the intermediate portion and the tissue fixing member are connected with each other by a fixing connecting structure in which the intermediate portion and the tissue fixing member are fixed with each other and integrated.

17. The treatment system according to claim 16, wherein
the fixing connecting structure includes:
one end of the tissue fixing member; and
a connecting concave portion formed at the intermediate portion, wherein the one end of the tissue fixing member is inserted into the connecting concave portion, and side surfaces of the one end of the tissue fixing member is sandwiched by the connecting concave portion.

18. An endoscope system comprising:
the treatment system according to claim 1; and
an endoscope having a channel into which the treatment system is inserted.
